(19) United States
(12) Patent Application Publication
(10) Pub. No.: US 2025/0325210 A1
(43) Pub. Date: Oct. 23, 2025

(54) SENSOR FOR DETECTING SURFACE ACOUSTIC WAVES IN FORCE MYOGRAPHY

(71) Applicant: New York Institute of Technology, Old Westbury, NY (US)

(72) Inventors: Fang LI, Great Neck, NY (US); Michael Christopher KOHLER, Flushing, NY (US)

(21) Appl. No.: 19/184,648

(22) Filed: Apr. 21, 2025

Related U.S. Application Data
(60) Provisional application No. 63/636,397, filed on Apr. 19, 2024.

(51) Int. Cl. A61B 5/22; A61B 5/00; G01L 1/16

(52) U.S. Cl. CPC A61B 5/221; A61B 5/6831; G01L 1/16; A61B 2562/0204; A61B 2562/0261

(57) ABSTRACT

A force myographic system utilizes a surface acoustic wave sensor to gather information about muscle activity to provide an estimate of torque provided by a muscle. The force myographic system includes the surface acoustic wave sensor, a conducting mounting bar and an adjustable band on which the sensor is mounted for application to a user's body to provide information indicative of torque provided by a muscle in the user's body.

### CROSS-REFERENCE TO RELATED APPLICATION

The present disclosure claims benefit of and priority to U.S. Provisional Patent Application Ser. No. 63/636,397, filed Apr. 19, 2024, entitled SENSOR FOR DETECTING SURFACE ACOUSTIC WAVES IN FORCE MYOGRAPHY, the entire content of which is incorporated by reference herein.

### BACKGROUND

### Field of the Disclosure

The present invention relates to a force myography system that uses a piezoelectric sensor to detect muscle movement. In particular, the system uses a surface acoustic wave (SAW) sensor device to determine muscle movement.

### Related Art

Accurate assessment of skeletal muscle forces and the resulting net joint torque may be used to prevent fatigue-related injuries, monitor changes in physical performance, and diagnose or manage neuromuscular conditions such as Parkinson's disease, multiple sclerosis, and muscular dystrophy. Precise torque measurements are especially valuable in sports medicine, physical therapy, and clinical rehabilitation, where they provide actionable insights into muscle performance and guide interventions to improve patient outcomes. For instance, a drop in muscle force output, and consequently, the torque produced at the joints during physically demanding tasks such as weightlifting or intensive manual labor, can indicate emerging fatigue. Recognizing these signals in real-time helps practitioners adjust an individual's training protocol, technique, or rest intervals to reduce the risk of overexertion-related injuries.

Routinely tracking torque provides valuable insights into the progression or improvement of neuromuscular conditions, where an increase in measured torque over time may signify a decline in disease severity or a positive response to therapy, while a decrease may point to worsening symptoms or the need for treatment adjustments. Traditional joint torque evaluation is performed in clinic-based settings to measure muscle strength, typically using an electromechanical dynamometer. This system is widely used for both isometric (fixed joint angle) and isokinetic (constant angular velocity) strength assessments. During isometric testing, the joint angle remains constant while the muscle exerts force against an immovable resistance, which outputs torque measurements without limb movement. In isokinetic testing, the dynamometer maintains a set angular velocity, allowing complete torque production profiles across the range of motion. Electromechanical dynamometry is known for its precise torque and angular position measurements, making it not only a clinically accepted standard for muscle performance evaluation, but also a research-grade gold standard. However, issues such as high costs, potential injury risks due to misuse and size constraints remain, making it crucial to conduct electromechanical dynamometry under controlled conditions to ensure accurate and safe evaluations.

Wearable systems for joint torque estimation have advanced considerably, aiming to reduce laboratory constraints and enable continuous, real-time monitoring. Early efforts primarily used surface electromyography (sEMG) to estimate muscle force or joint torque from muscle activity, but this approach is susceptible to errors that may result from electrode placement sensitivity, variable skin conductivity, motion artifacts, and high data/computational demands. Electrical impedance myography (EIM) tracks changes in muscle conductivity but is susceptible to hydration and skin-impedance fluctuations. Recent work shows combining EIM with sEMG may enhance torque estimation. Inertial measurement units (IMUs) derive joint torque from kinematics but are limited by drift and misalignment, especially at complex joints like the hip. Wearable ultrasound directly measures muscle morphology, yet it requires stable skin contact and remains vulnerable to motion artifacts and largely user dependent.

Physical therapy and sports science also benefit from an understanding of the biomechanics involved in limb movements and muscle activity to effectively evaluate physical performance and to customize training or interventions. In more specific applications like controlling exoskeletons and prosthetics, the real-time tracking of limb and muscle parameters like strain, force, and torque is increasingly essential.

Force Myography (FMG) provides a non-invasive method for assessing muscle function by monitoring volumetric changes in the musculotendinous complex (MC) or the resulting radial force distributions and overcomes shortcomings of electromyography, inertial measurement units, and ultrasound-based systems discussed above. By measuring surface-level muscle deformations arising from deeper volumetric changes, FMG provides benefits such as long-term signal stability, robustness against electrical noise, reduced sensitivity to sweating, low cost, and a donning-friendly setup. Existing FMG studies for joint torque estimation illustrate both promise and limitations. For instance, Sakr et al. utilized a multi-FSR (force-sensitive resistor) FMG band and three regression algorithms including general regression neural network (GRNN), support vector regression (SVR), and random forest regression (RF) to estimate isometric wrist torque to provide accuracy, however, requires bulky cables and on-board batteries. Moreover, these studies did not incorporate isokinetic trials.

Alvarez et al. introduced soft strain sensors and a cubic fit approach to estimate knee torque under isometric conditions and peak torque under isokinetic conditions. (J. T. Alvarez, L. F. Gerez, O. A. Araromi, J. G. Hunter, D. K. Choe, C. J. Payne, R. J. Wood, and C. J. Walsh, “Towards soft wearable strain sensors for muscle activity monitoring,” IEEE Transactions on Neural Systems and Rehabilitation Engineering, vol. 30, p. 2198-2206, 2022). However, this approach relied on wired power and established an isokinetic model solely from isometric data acquired at a single 90° joint angle. Marquardt et al. utilized barometric-based FMG arrays and Gaussian process regression (GPR) for isokinetic knee and ankle torque measurements, however also used a wired sensor configuration and restricted testing to a laboratory environment. (C. Marquardt, A. Schulz, M. Dezman, G. Kurz, T. Stein, and T. Asfour, “Force myography based torque estimation in human knee and ankle joints,” ArXiv, vol. abs/2409.11061, 2024. [Online]. Available: https://api.semanticscholar.org/CorpusID: 272693850). Further the small sample size (n=2) further limits the generalizability of this approach. Collectively, these studies emphasize FMG's potential for robust torque quantification, however, highlight the need for truly wireless, battery-free implementations to overcome practical constraints imposed by cabled setups and frequent battery maintenance.

Most studies focusing on FMG use force-sensing resistor (FSR) sensors, which are cost-effective and flexible but require consistent skin contact and can be prone to errors. As an alternative, resistive strain sensors, also commonly used, measure muscle deformation without needing skin contact, offering design flexibility and reliability, however, face challenges like non-linear responses and sensitivity to environmental conditions.

Monitoring limb movements and muscle activity across various anatomical regions is important in multiple disciplines, such as rehabilitation, sports science, and general wellness. Understanding biomechanics plays a pivotal role in the quantitative assessment and objective physical performance evaluation and facilitates the tracking of progress over time as well as enabling the customization of interventions or training regimens to meet individual requirements. In the context of biomedical applications, including exoskeletons and prosthetics, real-time monitoring of parameters such as strain, force, and torque resulting from limb movements and muscle activity has attracted significant attention from the scientific community.

As noted above, dynamometry is an essential tool in health sciences for measuring muscle or muscle group force during contraction using devices such as hand-held and isokinetic dynamometers. Hand-held dynamometers offer portability and simplicity, whereas isokinetic models, like the gold-standard Biodex Isokinetic dynamometer, ensure controlled speed during muscle contractions for consistent strength assessments. These techniques aid in diagnosing neuromuscular disorders, assessing muscle weakness, and monitoring recovery across various conditions. However, challenges such as high costs and potential injury risks due to misuse or size constraints exist. Conducting dynamometry in controlled settings is essential to ensure accurate and safe assessments, which limits mobility.

As mentioned above, surface electromyography (sEMG) and FMG are emerging as prominent techniques for muscle monitoring. sEMG measures the electrical signals generated by muscle contractions through electrodes placed on the skin, offering a non-invasive approach to assess muscle activity. However, as noted above, the reliability of sEMG poses challenges due to its vulnerability to various noise sources, including adjacent muscle crosstalk, improper electrode placement, movement artifacts, electromagnetic interference, ECG artifacts, and variations in skin temperature and humidity. Avoiding these issues requires meticulous attention in both sEMG hardware setup and the interpretation of its data which limits its usefulness.

As noted above, FMG provides an alternative, non-invasive technique for muscle function assessment by tracking volumetric changes within the musculotendinous complex (MC) or the resulting radially directed force distributions. Force or strain sensors may be embedded in a band, fabric, or adhesive patch placed over the targeted muscle to measure mechanical deformation during contractions, leveraging this deformation to estimate muscle force. FMG is more cost-effective, and there benefits from minimal effect of skin impedance, and high a signal-to-noise ratio (SNR).

Force-Sensing Resistor (FSR) sensors composed of a conductive polymer whose resistance decreases with applied force or pressure are typically used in FMG. Such FSR sensors are cost-effective, flexible, and robust, and do not require complex circuitry. However, as noted above a drawback of FMG is a requirement for close and consistent skin contact. The FSR sensors also experience certain static errors, including drift and sensitivity errors.

In alternative configurations, strain sensors, particularly resistive-based ones, may be used to measure muscle deformation for estimating force or torque without skin contact, thus avoiding some of the drawbacks of the FSR sensor and providing more flexibility in wearable designs, such as elastic bands, while maintaining high reliability during movement. Soft, stretchable strain gauges based on carbon fiber composites or carbon nanocomposites on knitted fabric may be used and offer high sensitivity, resilience, and wearability. Hysteresis, nonlinear electromechanical response, and sensitivity to temperature and humidity may limit the usefulness of such strain gauges.

The currently employed sensing techniques typically involve active sensors. In order to provide a wireless application, the sensor nodes would need to include not only the sensor but also silicon-based chips for powering the sensors, collecting and processing sensor signals, and wirelessly transmitting data to a mobile device. For instance, Gong et al. recently introduced a flexible wireless sEMG sensor system, incorporating a stretchable sEMG patch attached to the skin and a flexible printed circuit board (FPCB) worn on the arm. While this system enhances wearability and reduces noise, miniaturizing such a system presents challenges such as power constraints, substantial heat generation, and reduced communication distance.

Accordingly, it would be beneficial to provide a sensor for use in FMG that avoids these and other problems.

### SUMMARY

It is an object of the present disclosure to provide a surface acoustic wave (SAW) sensor to determine muscle movement.

It is a further object of the present disclosure to provide a SAW-based FMG technique for joint torque assessment.

It is also an object of the present disclosure to validate the SAW-based FMG technique against a gold-standard electromechanical dynamometer and to demonstrate accurate torque estimation at both isometric and isokinetic conditions.

A force myography system in accordance with an embodiment of the present disclosure includes: a surface acoustic wave sensor, the surface acoustic wave sensor includes, an SMA connector operable to provide an output of the surface acoustic wave sensor; an adjustable band configured for removable attachment to a user, wherein the acoustic wave sensor is mounted on the adjustable band such that movement of a muscle of a user is indicated by the output of the surface acoustic wave sensor.

In embodiments, the surface acoustic wave sensor includes comprises; a substrate including a first interdigital transducer and a second interdigital transducer; and a first open reflector and a second open reflector mounted on the substrate, wherein the SMA connector is operably connected to one of a first interdigital transducer and a second interdigital transducer.

In embodiments, the substrate includes a 500 μm thick, 128° YX-cut LiNbO₃ wafer.

In embodiments, the first interdigital transducer and second interdigital transducer comprise chromium and gold.

In embodiments, the force myography system includes a conducting bar, wherein the substrate is mounted on the conducting bar.

In embodiments, the conducting bar includes a 0.5 mm copper bar.

In embodiments, the force myograph system includes a terminal pad electrically connected to the first interdigital transducer.

In embodiments, the first interdigital transducer, second interdigital transducer, first open reflector and second open reflector are positioned in the substrate such that the first interdigital transducer uses an inverse piezoelectric output to convert electric signals into surface acoustical waves on the substrate which propagate along the substrate until they are reflected by the second interdigital transducer, the first open reflector and the second open back to the first interdigital transducer, wherein strain applied to the substrate by the muscle of the user delays receipt of the reflected waves at the first interdigital transducer corresponding to the torque applied by the muscle.

In embodiments the output of the surface acoustic wave sensor indicates a delay caused by the strain introduced by movement of the muscle.

In embodiments, the output of the surface acoustic wave sensor is processed using a 2D polynomial model to provide an estimate of the torque provided by the muscle.

In embodiments, the force myography system includes a controller operable to receive the output of the surface acoustic wave sensor and provide an indication of torque provided by the muscle.

In embodiments, the force myography system includes a wireless transmitter connected to the SMA connector and operable to transmit the output of the surface acoustic wave sensor wirelessly to the controller.

In embodiments, the controller is a computer system connected to a communications network.

In embodiments, the controller implements a 2D polynomial model to provide an estimate of the torque provided by the muscle.

In embodiments, the force myography system includes memory operably connected to the controller and configured to store the output of the surface acoustic wave sensor.

In embodiments, the memory further comprises controller executable code that when executed by the controller implements a 2D polynomial model to provide the indication of torque provided by the model based on at least the output of the surface acoustic wave sensor.

In embodiments, the indication of torque and the output of the surface acoustic wave sensor are stored in the memory.

In embodiments, the controller is operably connected to a display and displays the torque estimate and the output of the surface acoustic wave sensor.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates exemplary physical dynamics of a user's muscles during a bicep curl with a SAW sensor system in accordance with an embodiment of the present application in place on the user's arm;

FIG. 2 illustrates a more detailed view of a SAW sensor system positioned on a user's body to measure movement of the user's bicep including detailed views of a SAW component of the SAW sensor device in accordance with an embodiment of the present disclosure;

FIG. 2a illustrates Table I showing parameters used in the design of a SAW sensor in accordance with an embodiment of the present application;

FIG. 2b illustrates Table II showing assumptions used in conjunction with static biomedical equations;

FIG. 3a illustrates a block diagram showing reflecting bench testing of the SAW sensor in accordance with an embodiment of the present disclosure;

FIG. 3b illustrates examples of a user performing a preacher curl during testing of the SAW sensor in accordance with and embodiment of the present disclosure;

FIG. 4 illustrates a free body diagram depicting forces on the arm during the motion of the preacher curl;

FIG. 5a illustrates a time domain response of the SAW sensor after inverse Fourier transformation and zero padding of test data provided by the SAW sensor in accordance with an embodiment of the present disclosure;

FIG. 5b illustrates amplitude and phase response in the frequency domain of test data provided by the SAW sensor in accordance with an embodiment of the present disclosure;

FIG. 6 illustrates a normalized phase shift with strain changes using the bench testing strain apparatus during testing of the SAW sensor in accordance with an embodiment of the present disclosure;

FIG. 7a illustrates a comparison of SAW sensor phase shift to strain gauge response as a function of weight variation in accordance with an embodiment of the present disclosure;

FIG. 7b illustrates a phase shift in correlation to arm circumference in accordance with an embodiment of the present disclosure;

FIG. 7c illustrates normalized phase shift vs. strain variation with increasing weight in accordance with an embodiment of the present disclosure;

FIG. 7d illustrates a comparison of the SAW sensor shift to EMG sensor response as a function of weight variation in accordance with an embodiment of the present disclosure;

FIG. 8a illustrates Table III which illustrates recalibrated dynamic test data obtained by testing the SAW sensor in accordance with an embodiment of the present disclosure;

FIG. 8b illustrates comparative analysis of bicep force relative to weight during static preacher curl exercise at a 90-degree arm position;

FIG. 9 illustrates a comparative analysis of SAW, EMG, and strain gauge during dynamic preacher curl exercise during muscle loading;

FIG. 10 illustrates variation in microstrain and isometric hod normalize phase shift from dynamic testing;

FIG. 11 is a detailed view of an armband in which the SAW sensor system may be integrated in accordance with an embodiment of the present disclosure.

FIG. 12 illustrates a more detailed view of a SAW sensor system in accordance with an embodiment of the present disclosure;

FIG. 13 illustrates exemplary positioning of a SAW sensor system on a user's bicep in accordance with an embodiment of the present disclosure;

FIG. 14 illustrates exemplary positioning of a user during testing of the SAW sensor in accordance with an embodiment of the present disclosure;

FIG. 15 illustrates exemplary protocols for testing the SAW sensor system in accordance with an embodiment of the present disclosure;

FIG. 16 illustrates a table including test information from the testing protocols in FIG. 15 in accordance with an embodiment of the present disclosure;

FIG. 17 illustrates polynomial fits across angles of 15°, 30°, 45°, 60°, 75°, and 90° used to develop a model for use with the SAW sensor system in accordance with an embodiment of the present disclosure;

FIG. 18 illustrates a table showing isometric model performance information;

FIG. 19 illustrates 10°/s isokinetic trial for supinated elbow flexion, showing a time-series of joint angle, normalized phase shift, and measured versus predicted torque;

FIG. 19a illustrates normative torque profiles for supinated elbow flexion during concentric contractions at 10°/s;

FIG. 19b illustrates normative torque profiles for supinated elbow flexion during concentric contractions at 20°/s; and

FIG. 20 illustrates tables showing performance information at 10°/s and 20°/s respectively.

### DETAILED DESCRIPTION OF THE EXEMPLARY EMBODIMENTS

Surface acoustic wave (SAW) technology has been used for various sensing applications, including strain sensing, and offers passive operation and wireless interrogation via RF signals. In embodiments, a SAW sensor node may be worn on a user's body and may be chip-less, including only the SAW sensor and an antenna. In embodiments, the system may include a SAW sensor and a transceiver configured to transmit and receive information wirelessly. In embodiments SAW sensor design allows flexibility for high-sensitive strain sensing with good thermal stability. In embodiments, flexible SAW technology has become available and is suitable for wearable applications of SAW sensors. However, to date, there have been no studies or systems utilizing SAW sensors for FMG.

Surface acoustic wave (SAW) sensors offer wireless, battery-free operation alongside a high strain sensitivity, thereby paving the way for more seamless, real-world FMG integration. That is, SAW sensors may be used in an FMG system to provide information on the muscle performance. They leverage the inverse piezoelectric effect to generate acoustic waves along a piezoelectric substrate, where shifts in wave velocity or phase reflect external influences; radio-frequency interrogation then enables battery-free, wireless operation. Unlike traditional sensors used in FMG, SAW sensors also support flexible designs that can integrate multiple sensing modalities such as temperature, humidity, or strain in a single device, and when combined with functional polymer coatings, can serve as biosensors. Therefore, SAW techniques hold great potential for integrating multiple sensing capabilities, including muscle deformation, joint kinetics, temperature, and biochemical sensing, to advance wearable sensing applications.

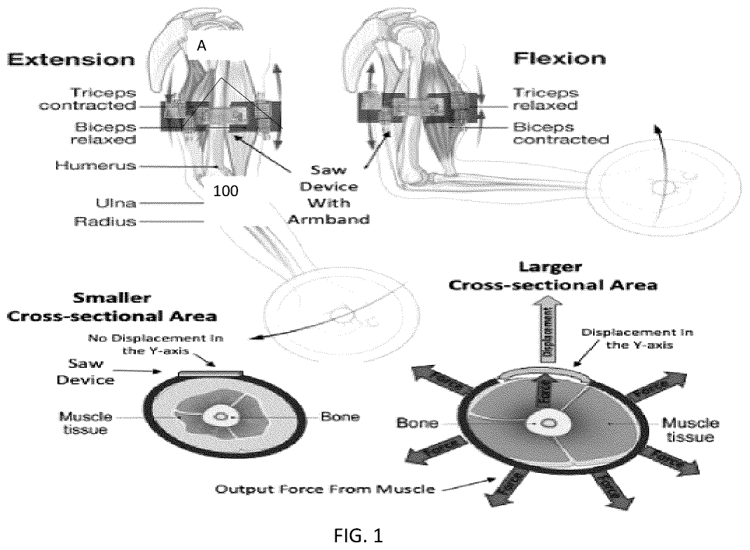

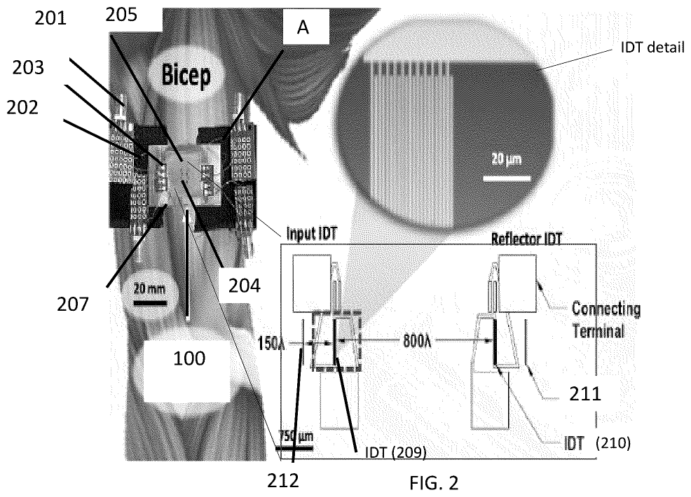

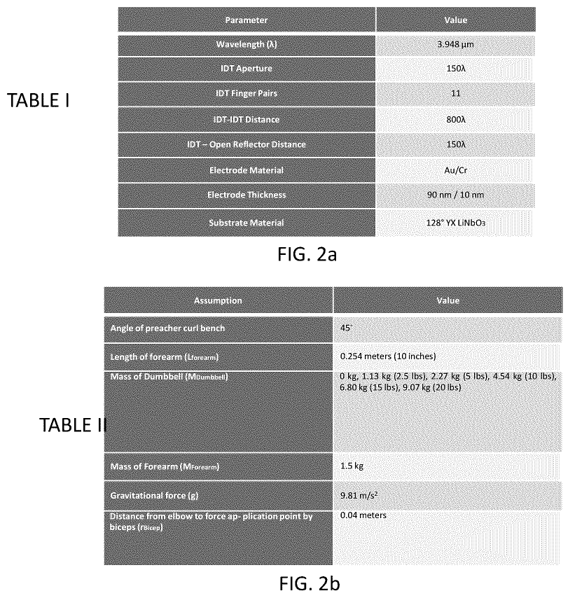

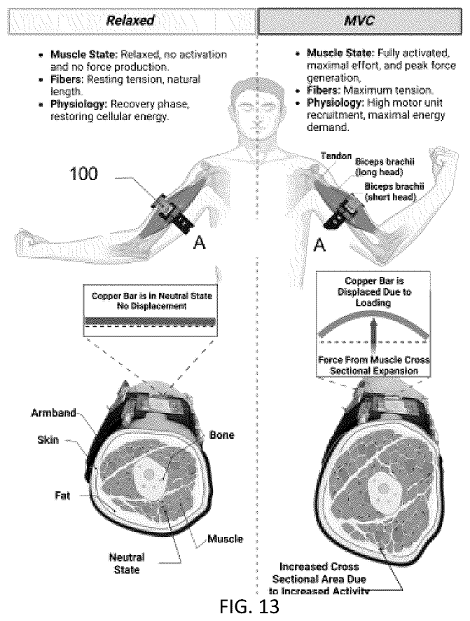

In embodiments, a novel SAW-FMG system 100 (see FIGS. 2 and 12, for example) provides enhanced detection of muscle contractions and relaxations. In embodiments, a wired setup and rigid sensor may be utilized however, as noted above, SAW sensors that simply include a sensor device 205 and antenna (or transceiver) that may be used to provide a wireless application. In embodiments, SAW sensor materials may be flexible which allows for development of wearable sensor devices that are comfortable and stable such that they can be integrated into an armband A (see FIGS. 2 and 12, for example) or another wearable element to position the sensor 205 or system 100 on a user's body. SAW sensor technology may be tested in analyzing the physiological dynamics of a standard bicep curl (see FIGS. 1 and 13, for example), employing a SAW sensor system 100 including an armband A securely fastened to the arm to detect volumetric changes in the muscle during contraction. Integrating the SAW sensor device 205 into the armband A as part of system 100 allows for positioning of the sensor and the system at any desired position along the user's arm. While the use of the sensor device 205 is described herein with respect to an arm curl, the sensor device 205 and system 100 may be positioned at other points on the user's arm to monitor movement of other muscles. In embodiments, the sensor device 205 may be integrated into another wearable element so positioning the sensor 205 with respect to other muscles in the user's body. In embodiments, testing of the SAW sensor 205 demonstrates its ability to quantify muscle force output under various loading conditions. In embodiments, when compared to traditional muscle activity detection methods, such as strain gauge measurements and conventional surface electromyography (sEMG), results provided by the SAW sensor device 205 confirm that such a sensor is a suitable replacement for strain gauges in muscle force measurements.

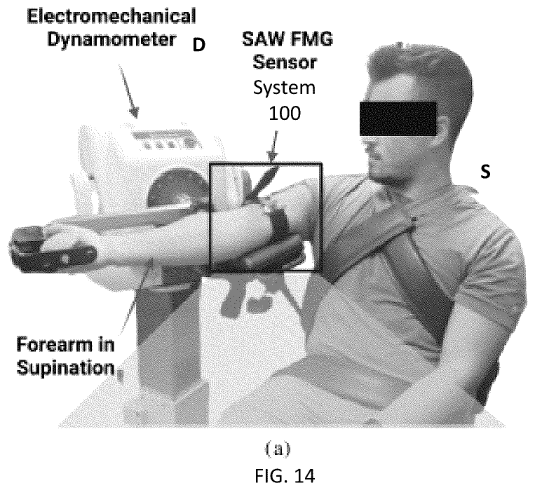

In embodiments, a SAW sensor 205, along with design and anatomical placement considerations, is illustrated in FIGS. 2 and 14. The relevant parameters used in testing are provided in Table I of FIG. 2A. In embodiments, the central frequency is designed as 915 MHz, and the delay line includes two interdigital transducers (IDTs) 209, 210 in FIG. 2 and two open reflectors 211, 212 (featuring multiple fingers). In embodiments, this configuration allows the device to function as a one-port or two-port sensor. In embodiments, the sensor 205 may be utilized as a one-port device, with one IDT (209) serving dual roles as both the input and output transducer. In embodiments, the second IDT 210, along with the two open reflectors 211, 212 may be used as reflectors. In embodiments, the input IDT utilizes the inverse piezoelectric effect to convert electric signals into the SAW on the substrate 204 (FIG. 2) of the sensor 205. These waves propagate towards the reflectors and are reflected, with returning waves converted back into electrical signals by the IDT 209. In embodiments, strain variations cause the crystal to expand or contract and alter the acoustic wave velocity. Consequently, the time delay between reflected signals increases or decreases accordingly. Thus, as the user's muscles move, they strain the crystal in a manner correlated to the muscle activity.

In embodiments, the sensor 205 may be fabricated using a 500 μm thick four inch 128° YX-cut LiNbO₃ wafer. In embodiments other wafers or materials may be used. In embodiments, after cutting the wafer to a desired size (15 mm×20 mm rectangles), the smaller pieces may be cleaned using acetone, isopropyl alcohol (IPA), and deionized (DI) water. In embodiments, other sizes may be used. In embodiments, other cleaning materials and processes may be used. In embodiments, the cut wafers may be bonded to a 0.5 mm-thick copper bar with adhesive. In embodiments, IDTs 209, 210 (see FIG. 2, for example) may be fabricated from chromium (Cr) as an adhesive layer and gold (Au), using lift-off process or any other suitable process. In embodiments, other methods of depositing the chromium or gold may be used and other deposition systems may be used. In embodiments, a combination of two different photoresists may be used, however, other processes may be used. A PMGI SF6 photoresist layer may be spin-coated at 3000 rpm for 90 seconds, followed by ZEP 520A under identical conditions. In embodiments, Espacer 300z may be spin-coated at 1500 rpm for 90 seconds for a total photoresist thickness of approximately 450 nm. In embodiments, patterns may be created using a JEOL e-beam writer at 10 nA current, 150 μc/cm2 dose, and 100 kV voltage. After that, in embodiments, the wafer may be prepared for development: the Espacer layer was rinsed with deionized water, the photoresist may be developed in Amyl Acetate, rinsed in IPA, further developed in ma-D 525, and finally washed with deionized water. The 10 nm Cr and 90 nm Au layers were sequentially deposited onto the wafer through thermal evaporation using the Kurt J. Lesker PVD 75 thin film deposition system, or any other suitable deposition system. Following the deposition, the wafers underwent a lift-off process to complete the procedure. In embodiments, other processes may be used.

After the SAW sensor 205 is fabricated, terminal pads 203 may be affixed onto the copper bar 207 using an adhesive. Subsequently, in embodiments, the SAW component 205 may be connected to these pads via a wedge wire bonder. In embodiments, for an initial strain test, the sensor 205 may be wired to an SMA (SubMiniature version A) connector 201 without a printed circuit board (PCB). In embodiments, however, the sensor 205 may be wired to a printed circuit board. In embodiments, while a particular structure of the SAW sensor 205 is illustrated, Applicant notes that SAW sensors may have different constructions and configuration and that any SAW sensor may be used on the system 100.

In embodiments, an adjustable blood flow restriction armband A may be tailored to desired dimensions, and the sensor 205 and accompanying components may be securely attached to it utilizing a fabric-specific adhesive. In embodiments, other arm bands or straps may be used to integrate and secure the sensor 205 to the user's body. In embodiments, printed circuit boards may be cut to the appropriate size and adhered to the armband A to support the attached SMA connectors 201 and these connectors may be soldered to the terminal pads using 30-gauge bare copper wire or any other suitable electrical connection. The sizes illustrate in FIGS. 2 and 12 are exemplary and other sizes and shapes may be used. In embodiments, other wires or connectors may be used. In embodiments, the armband A may be tailored to different sizes to allow for integration of the sensor 205 and application to muscles on other parts of the body. In embodiments, another wearable element may be used and the sensor 205 may be integrated therein for positioning the sensor 205 to monitor the movement of virtually any muscle.

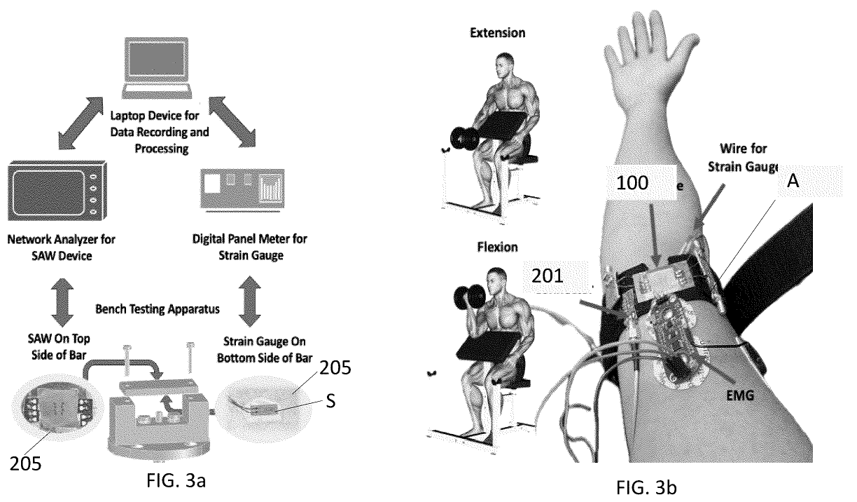

In embodiments, the SAW sensor 205 may be mounted on a custom-made fixed point load apparatus engineered to apply strain (see FIG. 3a). In embodiments, a strain gauge S may be affixed underneath the copper substrate to calibrate strain levels and may be connected to a gauge meter to provide precise measurements of the strain experienced by the SAW sensor 205 throughout the experiment. In embodiments, the strain gauge S is not necessary during normal operation.

In embodiments, the SAW sensor 205 may be connected to a Keysight E5061B-005 network analyzer as indicated in FIG. 3a. In embodiments, data may be recorded using one-port measurements. Therefore, in embodiments, the Si spectrum in the frequency domain may be recorded with a bandwidth of 250 MHz and a center frequency set to match the device's resonant frequency (915 MHz). Subsequently, in embodiments, a MATLAB program, or other software solution, may be employed to process the collected data. In embodiments, the raw data may be subjected to zero-padding before being transformed into the time domain using an inverse Fourier transformation. In embodiments, a time-gating technique may be applied to isolate individual pulses within the time-domain response. In embodiments, this process preserves data points corresponding to identified pulses while reducing the remainder of the time response to a near-zero level. The Fast Fourier Transform (FFT), in embodiments, may be employed to revert the truncated time-domain response to the frequency domain. In embodiments, the phase values at the central frequency of each pulse were determined and used to evaluate the resulting strain-induced phase shift. In embodiments, data was recorded for strain with the associated phase values ranging from 50 to 400 microstrain, in 50 microstrain increments.

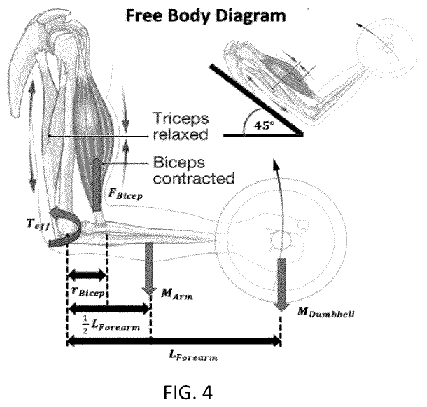

In embodiments, static biomechanical equations may be calculated to scale the output from the SAW sensor 205 to the corresponding bicep muscle force during a preacher curl exercise as indicated in FIG. 3b, for example, focusing on conditions where maximum torque occurs when the forearm is perpendicular to the bench's surface, angled at 45 degrees. In embodiments. these calculations may be used to accurately interpret and scale the SAW sensor data, reflecting the muscular force exerted in this specific exercise configuration. The assumptions for these calculations are included in Table II of FIG. 2B, and the free body diagram is illustrated in FIG. 4.

Given these parameters, the effective force (Feff) acting due to gravity on the dumbbell and forearm is computed as:

$$F ( eff ) = ( MDumbbell + 1 / 2 MForearm ) \cdot g \cdot cos ( 45 ° )$$

Subsequently, the torque (τeff) generated about the elbow joint by this force is:

$$τ eff = Lforearm \cdot Feff$$

The calculation for the bicep force (Fbiceps), necessary to hold the weight in position, is derived from the torque, factoring in the distance (rbiceps) from the elbow where the bicep's force is applied:

$${F}_{biceps} = \frac{{τ}_{eff}}{{r}_{Bicep}}$$

To accurately measure muscular activity in the upper arm, the custom SAW sensor 205, which is preferably wearable via the armband A may be affixed flush to the skin overlying the biceps brachii muscle using the armband. In embodiments, this placement allows for optimally registering the maximal deformation of the biceps during muscle contraction. A standard preacher curl exercise may be employed as the data collection method, as depicted in FIG. 3b. In embodiments, two separate testing approaches may be used: static and dynamic.

In the ‘static’ test, the SAW sensor 205 collects data at moments of maximum muscle contraction under varying weights, using a network analyzer for collection and MATLAB for processing. In embodiments, processing may be provided using other tools or software. In embodiments, a strain gauge and EMG sensors provide comparative measurements, affirming the correlation between changes in muscle strain and incremental load increases, however, the strain gauge and EMG sensors are not necessary during use of the sensor 205 or the system 100 in normal use. Throughout this test, the subject was positioned on the preacher curl exercise apparatus, maintaining a consistent arm posture. The data processing for the SAW sensor system 100 mirrored the methodology applied in the strain bench testing, focusing on analyzing the phase shift in response to the escalating load.

In embodiments, a baseline was marked by the forearm's perpendicular alignment (90 degrees) to the bench without bearing any weight, identified as the zero point. Measurements were then systematically taken at 0, 2.5, 5, 10, 15, and 20 pounds, with the strain gauge tared at this baseline for direct comparison. The SAW sensor's phase shifts may be calculated from this initial point. For comparison, the phase shifts were normalized by dividing by the number of wavelengths to achieve the phase shift per wavelength delay. The arm circumference was recorded at each weight step. This process aimed to analyze the correlation between muscle deformation under stress and changes in the phase response of the SAW sensor 205. Since the SAW sensor and strain gauge may be incorporated into the same armband A, simultaneous recording was possible. However, the EMG measurements may be conducted separately. This approach ensured that data collection from the EMG sensor remained isolated and unaffected by the presence of other sensors, guaranteeing accurate and non-interfering results. In embodiments, other steps or processes may be used.

In embodiments, EMG data was collected using a MyoWare sensor interfaced with a myDAQ, sampling muscle activity at 1000 Hz. The raw EMG data may be first bandpass filtered (20-450 Hz, 4th order Butterworth) to remove noise, then rectified for absolute muscle activation analysis. A 50 ms window RMS calculation was then applied to the rectified signal. Additionally, time-gating was used to identify specific lifting periods during the preacher curl exercise, focusing the analysis on relevant EMG segments. Subsequently, for each weight interval, standard deviations of the max peak EMG measurements were calculated. In embodiments, other techniques may be used.

In the ‘dynamic’ test, simultaneous use of both the SAW sensor 205 and system 100, strain gauge, and an electromyography (EMG) sensor attached to the subject's arm was used. In embodiments, data collection was executed via a network analyzer, gauge meter, and an myDAQ, with MATLAB being used for data recording and processing similar to that of the static test. In embodiments, other data collection techniques may be used.

In embodiments, data was recorded in the frequency domain at 100 points with a bandwidth of 40 MHz and a center frequency of 915 MHz. In this experiment, data acquisition rates may be varied across devices due to limitations of the hardware used: the strain gauge sampled at a rate of 1 Hz, the SAW sensor at 5 Hz, and the EMG sensor at 100 Hz. In embodiments, other rates may be used.

For the dynamic test, a specific baseline position was established whereby the forearm is parallel (180°) to the bench without any load, marking the zero point. The strain gauge was zeroed (tared) at this baseline, and the phase shift measured by the SAW sensor was normalized starting from this reference point. Subsequently, the subject was instructed to complete a single repetition of the curl with each specified weight (0, 2.5, 5, 10, 15, and 20 pounds) in succession, with approximately 5-second rest intervals between different weights. Additionally, to enable the simultaneous capture of data from all three sensors, both the strain gauge and SAW sensor were slightly offset by approximately 25 mm from the muscle belly or midpoint, moving towards the elbow. In contrast, in embodiments, the EMG may be offset by approximately 25 mm toward the shoulder (innervation zone) from the muscle belly. Pearson's correlation analysis may be conducted to assess the relationship between the data collected from the EMG sensor, strain gauge, and SAW sensor after both the SAW sensor phase data and the EMG RMS envelope were resampled to a 1 Hz frequency to align with the strain gauge.

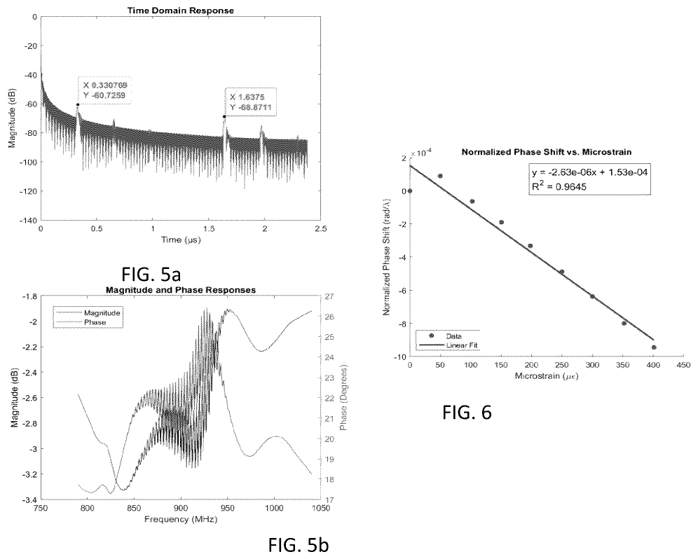

The time-domain response obtained post-inverse Fourier transformation and zero-padding, is depicted in FIG. 5a. The initial peak at 0.33 μs can be attributed to the signal reflection from a reflector situated 150A away from the input IDTs. The subsequent peak, noted at 1.64 μs, results from the reflection of the secondary set of IDTs, which are located 800A from the input IDTs. These observed delays align precisely with the sensor's theoretical design. The SAW sensor 100 was designed to attain its resonant frequency when the S₁₁ parameter's magnitude reaches a minimum, projected at 915 MHz. The amplitude and phase responses, illustrated in FIG. 5b, confirm that the resonant frequency approximates 915 MHz, in agreement with theoretical predictions.

A custom-designed strain apparatus was utilized to assess the sensor's initial performance under different load conditions prior to proceeding to the preacher curl exercise, which specifically engages the upper arm muscles to create the necessary loading conditions. FIG. 6 illustrates the normalized phase shift response to strain in the fabricated SAW system 100. The strain plotted is derived from strain gauge measurements. As the SAW sensor 205 and strain gauge were attached to the same spot but on opposite sides of the copper plate, it is presumed that they experience strains of equal magnitude but opposite signs. The Strain Coefficient of Delay (SCD), a measure of the sensor's sensitivity to strain, was calculated from this curve's slope given by the following equation:

$$SCD = ( \frac{slope}{2 π} ) \times {10}^{6}$$

This yielded an SCD value of −0.418 ppm/με for the fabricated sensor 100. This result presents an interesting comparison with related works. Specifically, Furniss et al. reported a higher SCD of approximately −0.57 ppm/με at room temperature. In contrast, Yan et al. documented a slightly lower SCD of −0.38 ppm/με, in their study of SAW sensors tailored for ambient temperature strain detection. The variability in SCD values contributes to factors such as substrate adhesives and testing configurations.

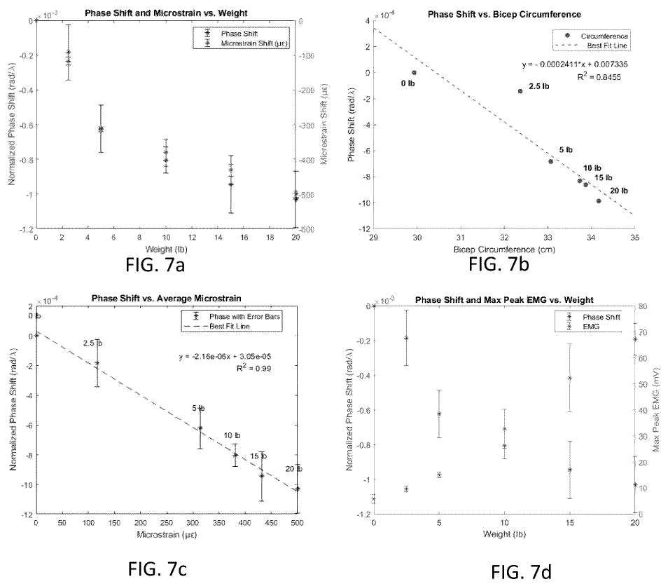

FIG. 7a compares signals from the SAW sensor 205 and the strain gauge under varying loads. Notably, the trends in the SAW sensor signal are similar to those of the strain gauge. The observed phase shift between the 0 lb and 5 lb load was −0.65×10⁻³ rad/λ, accompanied by a microstrain difference of −314με, indicating a notable initial muscle cross-sectional expansion in response to the applied load. However, the normalized phase shift for the load increase from 10 lb to 20 lb decreased to −0.2×10⁻³ rad, with a microstrain difference of −119με. This reduced responsiveness highlights a diminishing rate of muscle cross-sectional growth with higher loads, suggesting an upper limit to the extent of muscle cross-sectional expansion. This behavior illustrates the muscle's mechanical limits to expansion under increasing load, highlighting a critical consideration for sensor design and application in biomechanical studies. Prakash et al. also observed similar patterns using an FSR-based FMG system, highlighting that the sensor demonstrates greater sensitivity to lighter loads in comparison to heavier ones.

This observation was further confirmed by measurements of the circumference of the bicep brachii region during the static experiment. FIG. 7b shows the normalized phase shift versus variation in bicep circumference under different loading. The results reveal that the increase in muscle circumference is most significant within the initial 5 lb increase, showing a 3.1 cm expansion. Beyond this initial increase, the expansion becomes markedly less pronounced for subsequent weight intervals: a 0.67 cm increase from 5 lb to 10 lb, followed by a 0.13 cm and 0.3 cm expansion from 10 lb to 15 lb and 15 lb to 20 lb intervals respectively. The phase shift exhibits a linear relationship with the change in circumference of the bicep brachii region overall.

FIG. 7c shows the normalized phase shift response to the strain in the SAW system 100, similar to the preliminary bench testing. This result reveals a consistent slope trend in the defined microstrain dataset, though the exact strain intervals differ. In controlled bench testing, the SCD was determined to be −0.42 ppm/με, utilizing an apparatus that ensured uniform strain application for precise and consistent conditions. Conversely, during the static preacher curl exercise, a scenario introducing variable strain due to arm movement, the SCD altered to −0.34 ppm/με. This shift in SCD underscores the significant influence of the testing environment on SCD values, attributing the variation to the complexities of dynamic loads and muscle contractions encountered in real-world applications, in contrast to the controlled, predictable environment of bench testing.

In contrast to the strain and phase shift data, which showed the most notable changes during the initial 5 lb interval, EMG data demonstrated an inverse response pattern as depicted in FIG. 7d, with significant changes in output voltage occurring beyond the 5 lb mark. The transition from a 0 lb to a 5 lb load resulted in a modest EMG signal difference of 9.2 mV, marking the lowest increase among the measured intervals. Subsequently, increases from 5 lb to 10 lb and from 10 lb to 15 lb demonstrated more pronounced differences in the EMG signal, at 17.8 mV and 20 mV, respectively. This pattern underscores a marked escalation in muscular response beyond the initial 5 lb increment, suggesting a threshold of adaptation or engagement that becomes more significant with higher loads. This difference is attributed to EMG technology's reliance on muscle electrical potentials rather than cross-sectional expansion or stiffness. Suzuki et al. similarly observed that the mean absolute amplitude of surface electromyography rises in conjunction with greater force generation. They also concluded that the increase in mean absolute sEMG amplitude during a sustained contraction is dependent on the number of active motor units, their size, and their firing rates. Beretta et al. reported similar conclusions.

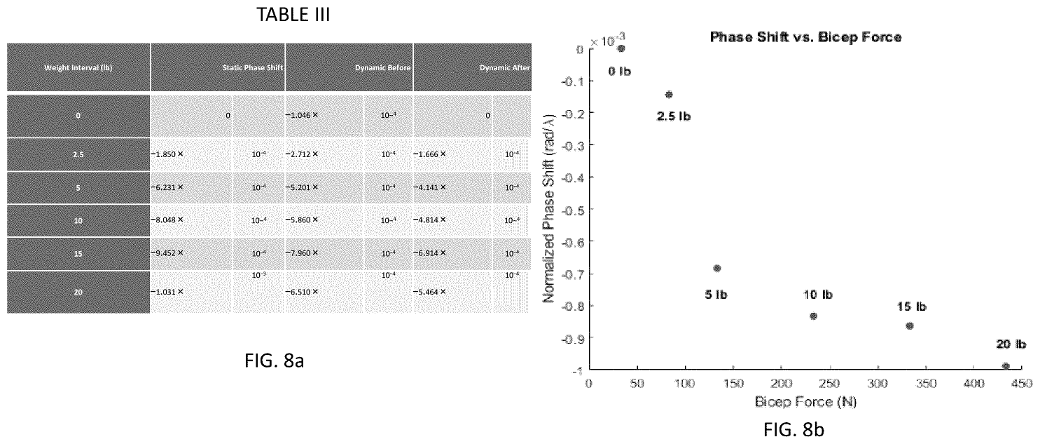

FIG. 8 illustrates the correlation between the observed phase shift and the expected force output from the bicep, as calculated from the previously outlined equations. This figure indicates that each specific force value generated by the bicep corresponds directly to a unique phase shift detected by the sensor.

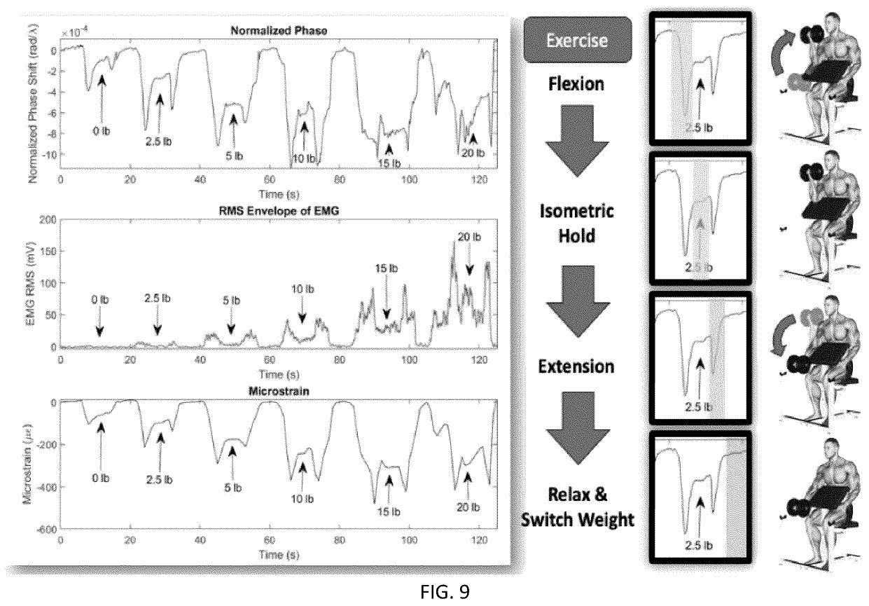

As depicted in FIG. 9, the test simultaneously captured the outputs from the SAW sensor 205 and system 100, EMG, and strain gauge sensor. The recordings distinctly display muscle contractions of varying intensities under different loads.

The visual comparison suggests that the SAW sensor system 100 is proficient at providing outputs over time that align with those derived from conventional methods, showcasing its potential for similar analytical performance in dynamic testing scenarios. The dataset reveals peaks during flexion (first peak) and extension (second peak) movements, illustrated in FIG. 9 (right), due to the brachialis muscle crossing the sensor array. Initially positioned on one side, the muscle then moves directly underneath the sensors and finally crosses to the other side. This path amplifies the strain and force exerted on the copper substrate of the SAW system 100 and strain gauge sensors precisely when the muscle is directly below them. Concurrently, the EMG sensor registers an elevated electrical output in this position, reflecting the muscle's maximum activity level. The distinctiveness of these peaks might be reduced by positioning the sensors at the anatomical midpoint of the upper limb. Such placement would ensure a more homogeneous distribution of the muscle's movement impact on the sensors, thereby potentially diminishing the variability of the readings. During this test, the sensors were positioned to allow simultaneous data collection, yet ideally, they should be placed at the anatomical midpoint of the upper limb.

The relationship between the outputs from the SAW sensor 205 and the strain gauge was analyzed, revealing a Pearson's correlation coefficient “r” of 0.790. This strong positive correlation is statistically significant, with a p-value of less than 0.001 in a two-tailed test, indicative of a significant association between the two datasets. Furthermore, the SAW sensor output, when compared with the EMG gauge output, demonstrated a Pearson's correlation coefficient of −0.550, revealing a moderate, statistically significant inverse relationship, affirmed by a p-value less than 0.001. Lastly, an analysis of the strain gauge and EMG gauge outputs yielded a Pearson's correlation coefficient of −0.569. This moderate negative correlation is supported by a statistically significant p-value of less than 0.001, highlighting a consistent inverse trend between the two measures.

The Pearson correlation coefficients of −0.550 and −0.569 for the phase and strain gauge comparisons with EMG, respectively, arise due to the initially lower responsiveness of the EMG at the beginning weight intervals of 0 lb and 2.5 lb. However, this responsiveness aligns more closely with the phase and strain measurements at the higher weight intervals of 5 lb, 10 lb, 15 lb, and 20 lb. This suggests that the EMG's sensitivity to muscle activation increases with the weight lifted, which is less apparent at lower weight levels when compared to the other two sensors.

In the comparative analysis of dynamic and static test outcomes, distinctions are primarily influenced by the initial arm and sensor positions. During dynamic evaluations, the arm's baseline position is parallel to the bench (180°), contrasting with the static scenario where the arm is perpendicular (90°). To align the baseline value of the dynamic test with that of the static test, the average reading from the dynamic test taken during the isometric hold at 0 lb is recalibrated to 0. This process entails modifying subsequent phase shift readings for each weight interval by deducting this baseline value from the mean value observed during the isometric hold phase. This adjustment ensures the initial conditions or baseline setpoint of the dynamic test are consistent with those of the static test for accurate comparison. This results in a correction of +1.046×10⁻⁴ for the 0 lb weight interval. The recalibrated dynamic test data is detailed in Table III of FIG. 8A.

Upon comparing individual data points for the distinct weight intervals, it is observed that the static test consistently yields lower phase shift values than their dynamic counterparts. This discrepancy stems from minor variances in sensor positioning on the arm across tests. Precisely, the static test's sensor placement near the muscle belly captures a larger cross-sectional area, leading to a more pronounced phase shift.

Conversely, the dynamic test positions the sensor closer to the myotendinous junction, registering a reduced cross-sectional area and, consequently, a less pronounced phase shift. Such sensor location differences also affect EMG signal outcomes in a similar manner.

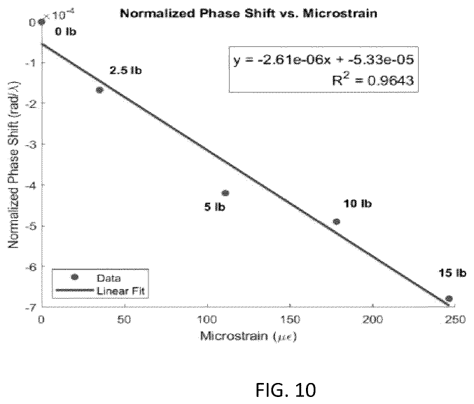

Furthermore, the data point at the 20 lb interval stands out as an outlier. This deviation may stem from the weight not being consistently held at the precise 90-degree angle, possibly positioned slightly lower, leading to an anomalously lower value than anticipated. By excluding this outlier and calculating the average value from the isometric hold, then plotting these adjusted values (FIG. 10), the slope can be calculated. From this analysis, the SCD for this adjusted dataset is −0.415 ppm/με, aligning with the findings from bench testing (−0.418 ppm/με) but slightly diverging from the static exercise experiment (−0.34 ppm/με).

The novel SAW sensor 205 for FMG provides a new approach for the detection and analysis of muscle contractions and relaxations. In embodiments the SAW sensor 205 may be integrated into an armband A in the SAW FMG system 100, which may be securely fastened to the arm of a user, enabling the detection of volumetric changes in the muscle during various stages of contraction and relaxation. In embodiments, armband A may be configured for placement of the SAW sensor 205 on different body parts. As noted above, the armband A may be positioned at different positions along the user's arm to allow for detection of muscle activity of different muscles. In embodiments, armband A may include an adjustment portion V that allows for the armband to secure the sensor 205 at different positions securely to provide accurate measurement. In embodiments, the adjustment portion V may include a hook-and-loop type fastener, for example. In embodiments, the armband A may be replaced by any other suitable wearable element that the sensor 100 is integrated into for secure positioning with respect to virtually any muscle in the body. The results of testing provided quantitative data on estimated muscle force output across a range of loading conditions for both static and dynamic states which supports the hypothesis that SAW sensors possess a unique sensitivity and accuracy in tracking muscle dynamics such that they are particularly well suited for use in determining muscle movement for FMG applications and appear to surpass the performance of traditional muscle activity detection methods such as strain gauge measurements and conventional EMG.

Further, the SAW sensor 205 may be implemented in a wireless application to fully utilize its passive characteristics to augment user comfort and facilitate uninterrupted monitoring. Integrating the sensor 205 into the armband A, or other wearable element, allows the sensor to be secured in a desires position for monitoring muscle activity such to further support passive monitoring of muscle activity.

Additionally, sensor configurations may be varied to accommodate various body regions will serve to heighten sensitivity and widen the system's applicability. In embodiments, the armband A may be modified or replaced with any suitable wearable element to provide secure and adjustable positioning of the sensor 205 with respect to other muscles.

Integrating the SAW sensor 205 in muscle activity monitoring systems may revolutionize rehabilitation, sports science, and biomechanical studies by providing a more accurate, reliable, and noninvasive method for assessing muscle function of virtually any muscle. This would open up possibilities for the development of advanced prosthetics and exoskeletons, where precise muscle force measurement is crucial. The incorporation of the sensor 100 into a wearable element, such as the armband A, further supports such applications.

In sum, the SAW system 100 includes the armband A which is adjustably securable to a user's arm as generally showing in FIG. 2 as well as the SAW sensor 205. As noted above, the armband A may be modified or replaced by any suitable wearable element to integrate the sensor 205 and allow for adjustable securement of the sensor to other parts of the user's body to allow for monitoring or virtually any muscle. The passive nature of the sensor 205, allows for passive and more or less continuous monitoring of muscle activity. The SAW sensor 205 may include piezoelectric (Lithium niobite, for example) substrate 204 mounted on copper plate (substrate) 207. A SAW component 206 may be provided on the substrate 204. In embodiments, the SAW component 206 includes two interdigital transducers (IDTs) 209, 210. IDT 209 acts as an input and the second IDT 210 acts as a reflector along with the reflectors 211, 212. As noted above, the IDTs 209, 210 may be constructed. A terminal pad 208 is connected to input IDT 209. In embodiments, an SMA connector 201 may be connected to terminal pad 203, which may be or be connected to terminal pad 208 connected to the IDT 209. In operation, input IDT 209 utilizes inverse piezoelectric effect to convert electric signals into SAWs on the substrate 204 (FIG. 2). These waves propagate towards the reflectors 210, 211, 212 and are reflected, with returning waves converted back into electrical signals by the IDT 209. In embodiments, strain variations caused by muscle movement in the arm on which the sensor system 100 mounted act on the substrate 204 to cause the crystal to expand or contract and to alter the acoustic wave velocity. Consequently, the time delay between reflected SAW signals increases or decreases in accordance with the movement of the muscle.

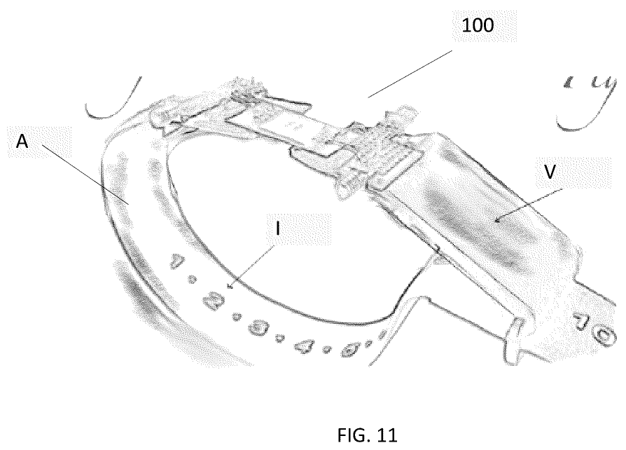

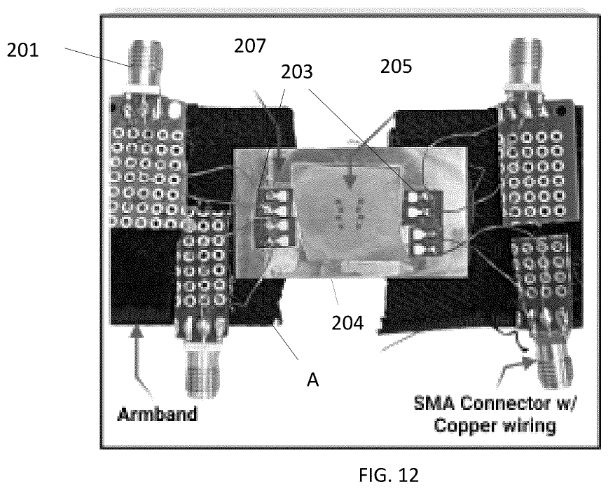

In embodiments, the armband A may include indicia I (see FIG. 11) which may be used for sizing purposes to provide a good fit. In embodiments, the armband A may include a hook-and-loop type fastener V, such as VELCRO which may be used to adjust the armband A, or other suitable wearable element, to allow for positioning of the sensor 100 with respect to virtually any muscle. The passive nature of the sensor 100 and its secure integration into the armband A or other wearable element allows for continuous monitoring of virtually any muscle.

FIG. 12 illustrates another view of the SAW system 100 including the SAW-FMG sensor 205 (see FIG. 12) and integrates a customized SAW delay-line sensor into the adjustable armband A to measure muscle activity. The system 100 is discussed above and common reference numbers are used to refer to common elements. In embodiments, contrary to conventional systems, the system 100 of the present disclosure uses a delay-line design for scalability, multiple sensors may be combined, and each delay line may encode a unique sensor identifier. In embodiments the SAW-FMG system 100 may have a center frequency of 915 MHz and may be derived from coupling-of-modes (COM) and finite element model (FEM) simulations. In embodiments, two interdigitated transducers (IDT's) 209, 210 and two open reflectors 211, 212 enable either one port or two port operations. As noted above, one of the IDTs may be used as a transmitter and receiver while the other IDS 210 and the reflectors 211, 212 are used to reflect the acoustic wave back to the IDT 209. That is, the system 100 if FIG. 12 uses the same SAW element 206 described above with respect to FIG. 2.

As noted above when an electrical signal is applied, the inverse piezoelectric effect excites acoustic waves that propagate, reflect, and then return to the same IDT (209), producing a time-delayed output based on wave propagation velocity. Strain-induced changes in the crystal lattice alter the wave propagation velocity, causing corresponding shifts in signal delay. That is, movement of the muscle may apply a strain in the crystal lattice of the substrate causing shift that corresponds to the force applied by the muscle. As is described further below, this delay may be associated with a torque provided by the muscle.

In embodiments, each sensor device 205 uses a 500 μm thick, 128° YX-cut LiNbO3 wafer 204 from MTI Corporation (Richmond, California, USA), patterned with IDTs 209, 210 made of chromium (Cr) and gold (Au) deposited through electron beam evaporation using a Kurt J. Lesker PVD 75 thin film deposition system (Jefferson Hills, Pennsylvania, USA). In embodiments, suitable wafers from other manufacturers may be used. In embodiments, the IDTs 209, 210 may be made using other techniques if desired. In embodiments, other film depositing systems may be used, if desired. In embodiments, the patterning process utilizes PMGI SF6 and ZEP 520A photoresists from MicroChem (Newton, Massachusetts, USA), with electron beam lithography performed using a JEOL JBX-6300FS system (Peabody, Massachusetts, USA). In embodiments, other photoresists and other lithography systems may be used. In embodiments, the wafers may be bonded to a 0.5 mm thick copper bar 207 from McMaster-Carr (Elmhurst, Illinois, USA) for structural support. In embodiments, other bars may be used and the thickness thereof may vary. In embodiments, electrical connections may be made through a Kulicke and Soffa wedge wire bonder (model 4526, Singapore) to SMA connectors soldered with 30-gauge copper wire. In embodiments, a different bonder may be used and different connectors may be used, In embodiments, for muscle performance assessment, the SAW sensor device 205 may be integrated into adjustable armbands A from Staminaa (Clermont, Florida, USA) using fabric-specific adhesives from Gorilla Glue (Cincinnati, Ohio, USA). In embodiments, any suitable fabric adhesive may be used and any suitable adjustable armband may be used. Signal acquisition and analysis may be performed using a Keysight Technologies network analyzer (model E5061B-005, Santa Rosa, California, USA) in some embodiments, however, other analyzers may be used, if desired. While the present disclosure describes a particular implementation of the SAW sensor 205, it is note that other configurations and embodiments, may be used. In embodiments, any suitable SAW sensor 205 may be used in the system 100.

As illustrated in FIG. 13, the sensor 205 has a delay line oriented perpendicular to the biceps brachii's longitudinal axis when the armband A is fastened around the bicep of a user, near the midpoint between its innervation zone and the myotendinous junction. In the relaxed state (left side of FIG. 13) minimal force is transmitted, however, during maximum voluntary contraction (MVC) (right side of FIG. 13), the muscle's significant expansion transfers strain to the attached sensor 205. This strain alters the acoustic propagation characteristics of the SAW sensor 205 thereby shifting its resonant frequency. In embodiments, a strain gauge (for example, a CFLA-3-350 from Tokyo Measuring Instruments Lab) may be provided to the underside of the copper bar 207 to ensure consistent tightening at approximately 55με across subjects. By tracking these resonance shifts in real time, the SAW-FMG sensor 205 offers a noninvasive, sensitive measure of muscle force output. In embodiments, output of the sensor 205 may be provided to a processor or control unit to provide an indication of the muscle activity. In embodiments, the processor or control unit may be external to the system 100. In embodiments, the output of the sensor may be transmitted wirelessly to a mobile computer device such as a smart phone, laptop or any other suitable device. In embodiments, the output of the sensor 205 may be transmitted over a wireless network to a server or to the cloud for storage and/or processing.

Isometric contractions may be used to establish a model relating sensor output from the sensor device 205 to biceps brachii-generated torque. Once the model is established, it may be used to predict elbow torque during concentric isokinetic flexion, which may be used to provide real-time torque estimation under realistic loading conditions. In embodiments, the model may be implemented in the server of control unit to provide the indication of torque based on the sensor output. As noted above, the sensor output may be provided to a mobile computing device, which may also use the output to provide an indication of the torque.

In embodiments, seven healthy participants (four males, three females; mean±SD: age 25.1±1.6 years, height 1.67±0.10 m, mass 75.5±23.1 kg) were recruited for an experimental protocol using sensor device 100. All participants reported moderate to high levels of physical activity, and data was collected from the right arm for each individual, regardless of dominance. Written informed consent was obtained from all participants, and the study was approved by the New York Institute of Technology Institutional Review Board under protocol NYIT IRB-2024-120.

Upon arrival, participants completed a warm-up session that included both static and dynamic upper-limb stretches. They performed arm-ergometer exercise for 5 minutes at 50% of their self-determined maximum effort and then for 5 minutes at 75% effort, for a total warm-up duration of 10 minutes. In embodiments, other warm up activities may be used.

Each participant was then fitted with the SAW-FMG system 100 discussed above. Participants were then seated at an electromechanical dynamometer D, and the dynamometer and positioning chair C were adjusted to align the elbow joint with the dynamometer's axis of rotation. In embodiments, chest straps S may be applied to minimize trunk movement. A calibration procedure may be conducted to establish a range of motion (ROM), with safety stops at both extremes. Finally, to familiarize themselves with the testing protocol and the SAW-FMG system 100, participants performed a series of submaximal isometric and isokinetic elbow flexion contractions of the biceps brachii with the forearm in a fully supinated position as is illustrated in FIG. 14. The experimental setup is illustrated in FIG. 14.

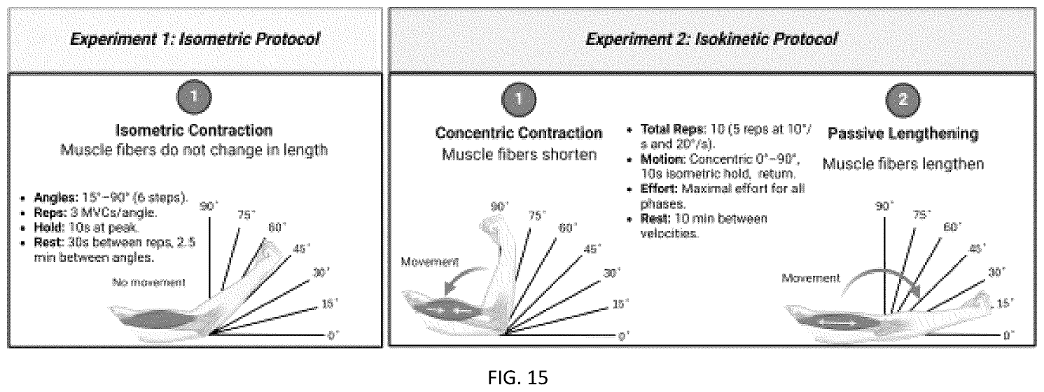

The participants performed a total of 10 isokinetic repetitions in a fully supinated forearm posture, including 5 maximal-effort trials each at angular velocities of 10°/s and 20°/s. Each repetition began with a concentric contraction from 0° to 90° of elbow flexion, followed by a 10-second isometric elbow flexion hold at 90°. This hold attempted to accommodate potential latency in the SAW sensor device output and to assess system limitations. Participants then guided the arm back to 0°, allowing the biceps brachii to lengthen passively since only the concentric phase was analyzed in this study. A 5-second rest period was then included after passive lengthening again to accommodate potential latency in the SAW sensor device output. There was a 10-minute rest period between each set of five repetitions at a given velocity. The experimental protocol for isometric testing and isokinetic testing are illustrated in FIG. 15.

In both experimental configurations, the electromechanical dynamometer D measures torque and angle at a sampling frequency of 100 Hz. In contrast, the SAW-FMG sensor 205 samples at 2 Hz due to limitations of its hardware. The difference in sampling frequencies is considered in the following sections, which describe the methods used for data synchronization.

At each selected isometric angle, three repetitions were recorded per subject. The rising phase of each isometric contraction, starting from the onset of contraction to peak torque, was extracted from both the dynamometer and sensor data. One-port_{Σ11} was measured, and the phase shift φ(τ) was derived to quantify strain, as detailed by Kohler et al., The phase shift and torque signals were then time-normalized to the interval [0, 1], standardizing the duration of the rising portion across repetitions.

To account for temperature-related offsets in the sensor, each repetition's phase shift data at each angle was shifted so that its waveform began at zero. If ϕ(t) denotes the original phase waveform, the shifted phase is:

$${ϕ}^{′} ( t ) = ϕ ( t ) - ϕ ( 0 )$$

ensuring that ϕ′(0)=0 at the onset of contraction. Following this offset correction, torque measurements at discrete time points were interpolated onto the sensor's normalized time base, yielding synchronized phase-torque pairs. Next, the phase and torque waveforms were min-max normalized so that the least negative sample of each waveform mapped to 0 and the most negative mapped to −1. Hence, each subject and each isometric angle θ contributed three repetitions of matched phase ϕ and torque τ samples (both in [0, −1].

A two-step procedure was utilized to relate ϕ, θ and τ. First a 1D polynomial fit (ϕ to τ) was carried out at each angle θ by combining all repetition data (three reps ×tjat subject. A second-order polynomial:

$$τ ( ϕ ) = {p}_{0} + {p}_{1} ϕ + {p}_{2} {ϕ}^{2} ,$$

was fitted via least squares. Similarly, a group-level 1D polynomial was obtained for each angle by pooling all subject's data. Next, to form a 2D polynomial τ(θ, ϕ), each subject's angle-wise 1D fits were sampled across ¢ values, creating a bivariate dataset {θi, ϕi, τi}. Following the second order model:

$$τ ( θ , ϕ ) = {α}_{00} + {α}_{10} θ + {α}_{01} ϕ + {α}_{20} {θ}^{2} + {α}_{11} θ ϕ + {α}_{02} {ϕ}^{2} ,$$

was then fitted.

Let ⊖i and Φi denote the angle and normalized phase shift, and let τi be the normalized torque. If a subject has N total samples across all angles (after sampling the 1D fits), then the design matrix Xsubject, coefficient vector α, and torque vector τ subject are:

$${X}_{subject} = [ \begin{matrix}1 & {θ}_{1} & {ϕ}_{1} & {θ}_{1}^{2} & {θ}_{1} {ϕ}_{1} & {ϕ}_{1}^{2} \\ 1 & {θ}_{2} & {ϕ}_{2} & {θ}_{2}^{2} & {θ}_{2} {ϕ}_{2} & {ϕ}_{2}^{2} \\ ⋮ & ⋮ & ⋮ & ⋮ & ⋮ & ⋮ \\ 1 & {θ}_{N} & {ϕ}_{N} & {θ}_{N}^{2} & {θ}_{N} {ϕ}_{N} & {ϕ}_{N}^{2}\end{matrix} ] ,$$

$$α = [ \begin{matrix}{α}_{06} \\ {α}_{10} \\ {α}_{01} \\ {α}_{20} \\ {α}_{11} \\ {α}_{02}\end{matrix} ] , {τ}_{subject} = [ \begin{matrix}{τ}_{1} \\ {τ}_{2} \\ ⋮ \\ {τ}_{N}\end{matrix} ] .$$

Subject level coefficients α are then obtained by least squares

$$α = {( {X}_{subject}^{T} {X}_{subject} )}^{- 1} {X}_{subject}^{T} {τ}_{subject} .$$

Group level dataset with M samples:

$${X}_{group} = [ \begin{matrix}1 & {θ}_{1} & {ϕ}_{1} & {θ}_{1}^{2} & {θ}_{1} {ϕ}_{1} & {ϕ}_{1}^{2} \\ 1 & {θ}_{2} & {ϕ}_{2} & {θ}_{2}^{2} & {θ}_{2} {ϕ}_{2} & {ϕ}_{2}^{2} \\ ⋮ & ⋮ & ⋮ & ⋮ & ⋮ & ⋮ \\ 1 & {θ}_{M} & {ϕ}_{M} & {θ}_{M}^{2} & {θ}_{M} {ϕ}_{M} & {ϕ}_{M}^{2}\end{matrix} ] ,$$

$${τ}_{subject} = [ \begin{matrix}{τ}_{1} \\ {τ}_{2} \\ ⋮ \\ {τ}_{M}\end{matrix} ] .$$

A global coefficient β is then derived via:

$$β = {( {X}_{group}^{T} {X}_{group} )}^{- 1} {X}_{group}^{T} {τ}_{group} .$$

This procedure produces both subject-specific and group-level coefficients for the two-dimensional polynomial characterizing normalized torque t as a function of angle ⊖ and normalized phase shift Φ. Once these coefficients are obtained, each model (subject-specific and group-level) was applied across each repetition for all angles (0-90°) for each subject, enabling a direct comparison of model performance. The mean coefficient of determination P² and mean root-mean-square error (RMSE) were calculated for the three isometric contractions to assess predictive accuracy. RMSE is defined as the square root of the average of the squared differences between the model's predictions and actual measurements. The coefficient of determination P² measures the proportion of the variance in the data explained by the model, with 1 representing a perfect fit and 0 indicating no improvement over the mean [45]. Since torque values were normalized before analysis, the reported RMSE is effectively a normalized root-mean-square error (NRMSE).

Next, single-factor repeated-measures ANOVA (Analysis of Variance), a statistical method for comparing group means across multiple conditions, was performed to determine whether the subject-specific model differed significantly from the group model in terms of average NRMSE and P² across all angles. Because this design involved only two repeated levels, sphericity, the assumption that variances of the differences across repeated conditions are equal, was inherently satisfied; therefore, Mauchly's test, a statistical procedure that detects violations of sphericity, was largely a formality. Normality was assessed by conducting a Lilliefors test, a modified Kolmogorov-Smirnov procedure for checking normality, on the paired differences (for example, SpecNRMSE-GroupNRMSE) and by examining quantile-quantile (Q-Q) plots. Which are plots that visually compare the data's distribution to a theoretical normal distribution. If the data exhibited a significant departure from normality (π<0.05), a Wilcoxon signed-rank test, the nonparametric analog of a paired τ-test, was used as a nonparametric alternative to the repeated-measures ANOVA. Statistical significance was set at α=0.05.

The raw isokinetic data included concurrent torque and angle measurements from the dynamometer and the phase shift signals from the SAW sensor 205. Only maximum voluntary (active) torque was recorded, with no passive tension or torque measured. To provide temporal consistency among these measures, the angle and torque data were interpolated to the time points of the phase shift signal, thus establishing a common time reference.

Having synchronized the data, a phase shift signal was normalized into a defined range of [0, 1]. The torque data underwent a similar transformation, mapping the least negative torque to 0 and the most negative torque to −1. This kind of transformation enabled direct comparison between the phase and torque signals on a shared numerical space.

Next, the polynomial model, initially calibrated during isometric trials, was used to predict normalized torque from the normalized phase and the measured joint angle. Mathematically, this torque prediction for each time point τ was expressed as:

$${\tilde{T}}_{norm} ( t ) = {β}_{00} + {β}_{10} θ ( t ) + {β}_{01} {ϕ}_{norm} ( t ) + {β}_{20} {θ ( t )}^{2} + {β}_{11} [ θ ( t ) {ϕ}_{norm} ( t ) ] + {β}_{02} {{ϕ}_{norm} ( t )}^{2} ,$$

where θ (τ) is the measured joint angle (in degrees), φnorm (τ) is the normalized phase, and βi are group-based coefficients obtained from the isometric calibration. Substituting the measured angle and normalized phase signals into this model yielded a continuous estimate of torque throughout the isokinetic trials, enabling a direct comparison between measured and predicted torque during dynamic muscle contractions.

Because this study focused exclusively on the concentric phase of the bicep's contraction, data from the isometric hold and passive lengthening phases were excluded. In embodiments, additional data may be used. Specifically, time intervals corresponding to those phases were identified, and their phase shift and torque values were set to zero. Five repetitions were performed at each angular velocity, and within each repetition, the concentric phase was analyzed cycle-by-cycle from 0° to 90°. This ensured that all subsequent analysis centered on the portion of motion actively driven by the biceps, consistent with the sensor's placement.

For each subject's concentric cycles (10°/s or 20°/s), NRMSE and Pearson correlation were computed between predicted and measured torque. Pearson's correlation coefficient (φ quantifies the linear relationship between two variables, ranging from −1 (perfect negative correlation) to 1 (perfect positive correlation), with 0 indicating no linear relationship. Means and standard deviations across cycles quantified overall performance. The best cycle was identified by minimal NRMSE, as mentioned earlier. Group mean±standard deviation for NRMSE and Pearson was then compiled across all cycles and best cycles, highlighting overall accuracy and best-case performance.

A one-way repeated-measures ANOVA was applied to both the NRMSE and Pearson correlation data for the 10°/s and 20°/s conditions, treating velocity as a within-subject factor across seven participants. Because only two levels of velocity were examined, sphericity was inherently satisfied. Normality was assessed by applying a Lilliefors test to the paired differences (e.g., NRMSE10-NRMSE20) and by visually inspecting quantile-quantile (Q-Q) plots. If the normality assumption was violated (π<0.05), a Wilcoxon signed-rank test replaced the repeated-measures ANOVA as a nonparametric alternative. This approach was repeated for both NRMSE and Pearson correlation measures, and the significance level was set at α=0.05 for all tests.

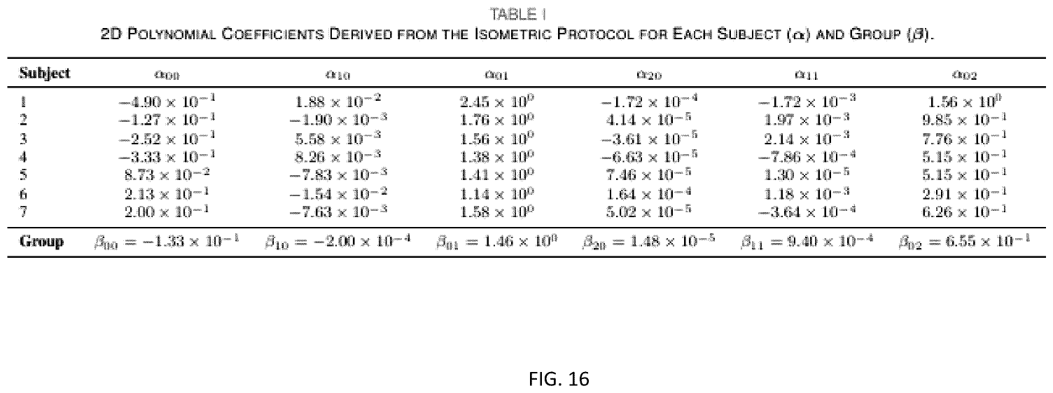

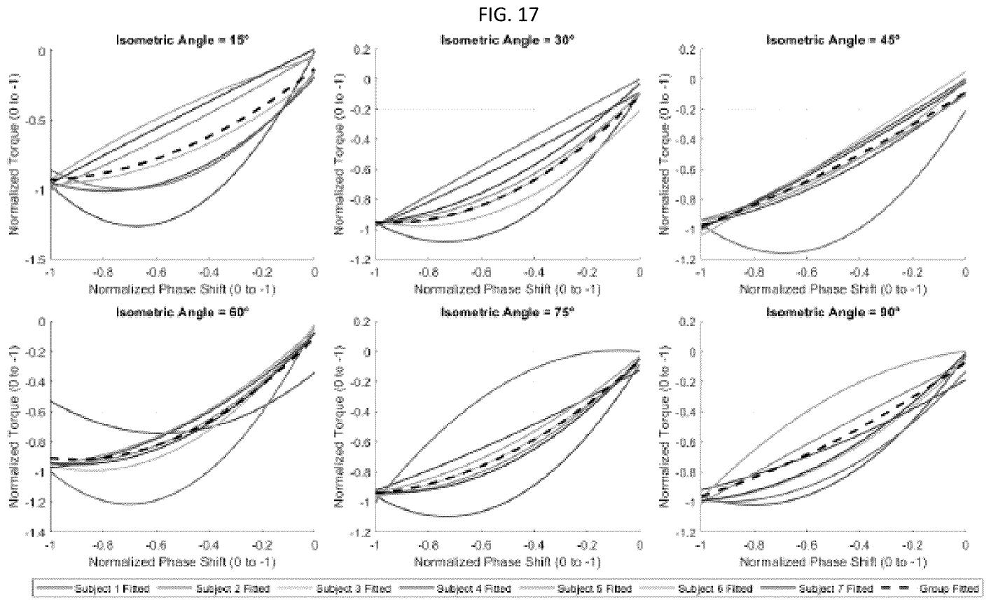

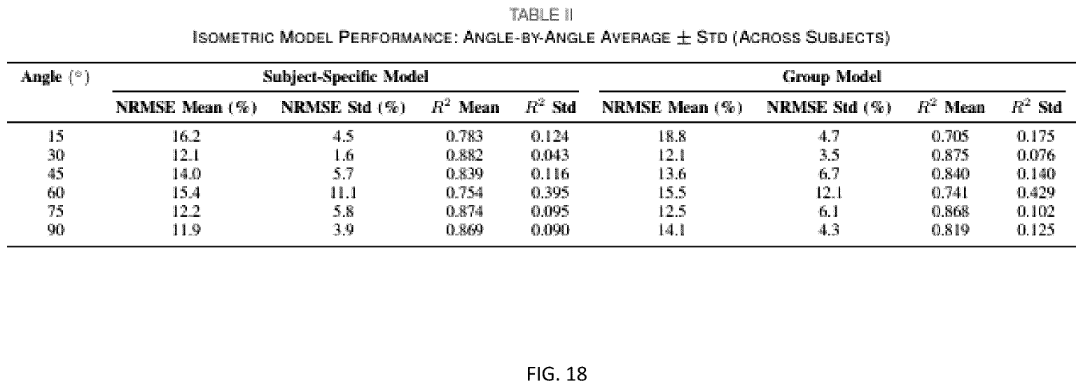

FIG. 17 illustrates the 1D (phase to torque) polynomial fits across angles of 15°, 30°, 45°, 60°, 75°, and 90°. Each subplot shows both the subject-level fits (one curve per subject) and the group-level 1D polynomial. Building on these angle-specific polynomials, a second-order bivariate model was then derived to relate joint angle, normalized phase shift, and normalized torque. The resulting model coefficients, for each subject and for the group, appear in Table I illustrated in FIG. 16. Using each subject's own coefficients across all angles (the subject-specific model) yields an NRMSE=13.6%±6.0% and P²=0.834±0.180 (mean±SD). In contrast, applying group-level coefficients produces NRMSE=14.4%±7.0% and P²=0.808±0.208, indicating that although a universal solution is obtained, subject-specific calibration provides a slightly higher overall accuracy. Table II presents the average NRMSE and □2 per angle for both modeling approaches. At 15°, 30°, 60°, 75°, and 90°, the subject-specific model consistently achieves lower NRMSE and higher P², along with smaller standard deviations reflecting more uniform performance across participants. As an example, at 15°, the subject-specific approach attains an NRMSE of 16.2%±4.5% compared to 18.8%±4.7% for the group model, and an P² of 0.783±0.124 versus 0.705±0.175. Notably, 45° is the only exception, where the group-level model slightly outperforms the subject-specific approach, likely due to this mid-range angle presenting more uniform torque generation across participants, thereby reducing inter-subject variability.

A single-factor repeated-measures ANOVA comparing the subject-specific versus group models (averaged across angles) revealed that the subject-specific model achieved significantly lower NRMSE than the group model [Φ (1,6)=10.75, π=0.017], although the numerical difference averaged only 0.8% across all angles. The Lilliefors test on the NRMSE differences (SpecNRMSE-GroupNRMSE) indicated no violation of normality (π=0.217), so no nonparametric alternative was required. Similarly, a repeated-measures ANOVA on P² 0.014], with the Lilliefors test (π=0.500) also confirming showed a significant model effect [Φ (1,6)=11.83, π=no departure from normality in the (Spec R²-Group R²) differences. Hence, while both the NRMSE and R² metrics demonstrated statistically reliable superiority for the subject-specific model, the absolute performance gains were modest.

Relative to prior work using diverse sensors and modeling strategies, the current approach exhibits comparable normalized errors. Alvarez et al. reported a mean NRMSE of 7% (rising to 9% with a generalized model) for 90° isometric knee flexion using soft wearable strain sensors validated against an isokinetic dynamometer. Youn et al. employed mechanomyography (MMG) and a feedforward neural net-work to achieve a 13.1% NRMSE in isometric elbow flexion, referencing a one-directional force-transducer [51]. Menegaldo et al. used a Hill-type EMG-driven model to estimate knee torque at 80° of knee flexion, validated against an isokinetic dynamometer, reporting NRMSE values of 15.8% and 29.5% at 60% and 20% of maximum voluntary contraction, respectively. Overall, these studies demonstrate state-of-the-art accuracy ranges, while discrepancies between them and the current study can largely be attributed to differences in experimental protocols, sensor working principle, estimation method, and the choice of ground-truth measurement.

The slight improvement in performance of the subject-specific model can be attributed to the fact that it is calibrated for each individual, thereby accounting for differences in muscle mass and fat distribution. By contrast, a group model provides a generalized solution that does not capture these variations. Prior research indicates that skin and subcutaneous fat dampen the forces from muscle fibers as they transmit to force-sensing resistors (FSRs), suggesting that higher adipose tissue may further attenuate FMG signals. Additionally, exact sensor placement often varies across subjects, as it is unrealistic to assume that FMG sensors can be applied to the exact same anatomical location for each user. In this study, we attempted to standardize sensor placement by having the subject flex the biceps brachii, identifying the muscle's peak point. The SAW system 100 was then positioned near the midpoint between its innervation zone and the myotendinous junction. Furthermore, every individual's muscle contracts with a unique shape, resulting in inherently distinct FMG signal patterns. Previous literature has also reported similar findings regarding the advantage of subject-specific calibration over group-level approaches. For instance, Wang et al. demonstrated that personalized EMG-driven torque models significantly reduce estimation error compared to a generalized model, while Alvarez et al. showed a similar trend in soft sensor-based knee torque. (H. Wang, B. Bardizbanian, Z. Zhu, H. Wang, C. Dai, and E. A. Clancy, “Evaluation of generic emg-torque models across two upper-limb joints,” Journal of Electromyography and Kinesiology, vol. 75, p. 102864, April 2024).

In addition to torque estimation metrics, the time to peak torque was evaluated for both the electromechanical dynamometer and the SAW-FMG system 100. Across all subjects, repetitions, and angles, the electromechanical dynamometer demonstrated a mean time to peak of 1.48±1.29 s (mean±standard deviation), whereas the SAW-FMG sensor 100 showed a time to peak torque of 3.43±1.50 s. The longer time-to-peak values observed with the SAW-FMG system 100, compared to the dynamometer reference, may be attributed to several factors. First, the SAW-FMG measurement captures volumetric muscle changes indirectly through the skin and subcutaneous fat, potentially introducing a slight lag before peak torque signals are registered.

In addition, the overall complexity of the data acquisition chain, from the SAW system 100 through the network analyzer N and onward to the computer running the MATLAB program, can further contribute to latency. Complex sensor systems often exhibit increased latency due to multiple processing stages and communication links required for data handling. Specifically, the network analyzer N must sample and digitize the sensor signal, communicate the measured amplitude/phase data to the host computer via USB, and then wait while the MATLAB software processes the incoming data. Each of these steps adds a small but non-negligible delay that increases the observed time to peak. It is noted that in operation, the system 100 need not be connected to the network analyzer as the output may be provided to a processor or controller that uses the model derived using the testing protocols discussed above.

Despite the extended signal path, the SAW-FMG system 100 continued to yield reliable torque estimates, as evidenced by its competitive normalized error metrics and strong correlation with the dynamometer reference. Subject-specific calibration further enhanced performance by accounting for variations in muscle mass, fat distribution, and sensor placement. However, to increase applicability across diverse user populations, a group-level model was adopted for subsequent isokinetic experiments. Even under these generalized conditions, the SAW-FMG system 100 still provided accurate torque estimation, under-scoring its potential for practical and scalable implementation.

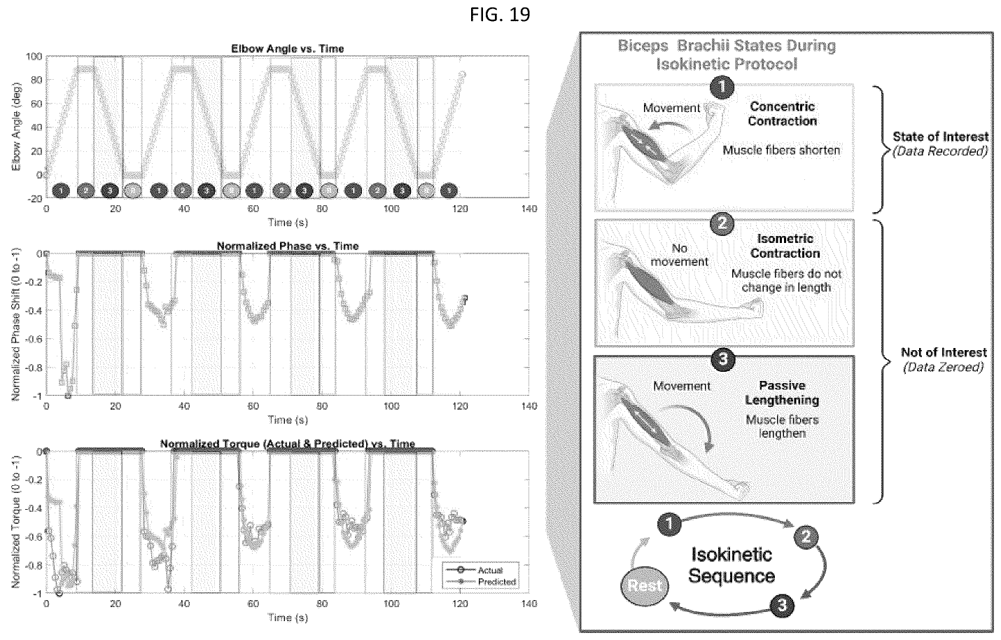

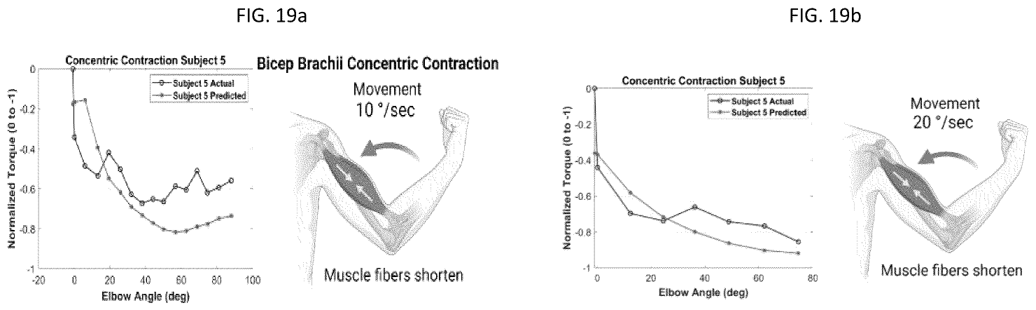

Investigating the model's performance under two distinct isokinetic velocities (10°/s and 20°/s) was motivated by the need to understand whether a single SAW-FMG sensor 205 could reliably adapt to dynamic muscle contractions. Specifically, this study sought to determine if the same calibration and polynomial model used in isometric tasks could be extended to controlled isokinetic movements with minimal loss in accuracy. FIG. 19 illustrates a representative 10°/s isokinetic trial for supinated elbow flexion, showing the time-series of joint angle, normalized phase shift, and measured versus predicted torque. Each cycle includes four biceps brachii states: concentric, isometric, passive lengthening, and rest, yet only the actively driven concentric phase is retained for analysis. FIGS. 19a and 19b present an example from one subject (Subject 5), showing the best cycles recorded at 10°/s and 20°/s, respectively.

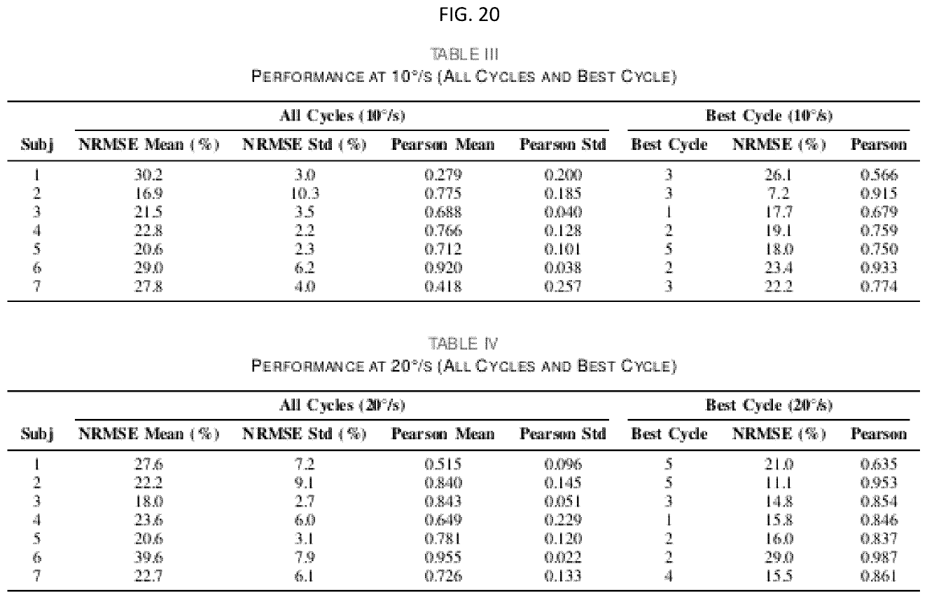

Tables III and IV in FIG. 20 summarize the resulting performance metrics from the two subjects, including the mean and standard deviation (std) of NRMSE and correlation r (across all cycles), along with the best cycle (lowest NRMSE). At 10°/s (Table III), the NRMSE across all cycles ranged from 16.9% to 30.2%, with r values between 0.279 and 0.920. The best cycle typically yielded a lower NRMSE (e.g., 7.2%) and higher r (up to 0.933). Likewise, at 20°/s (Table IV), the NRMSE spanned from 18.0% to 39.6% (all cycles) and from 11.1% to 29.0% (best cycle), while r rose as high as 0.987. Overall, across all cycles and subjects, the mean (±std) NRMSE is 24.1% to 6.6% at 10°/s and 24.9% to 8.8% at 20°/s, with corresponding r values of 0.651±0.252 and 0.758±0.179. Focusing only on the best cycle per subject reduces the NRMSE to 19.1%±6.1% at 10°/s and 17.6%±5.8% at 20°/s, while increasing r to 0.768±0.128 and 0.853±0.112, respectively.

A one-way repeated-measures ANOVA was applied to NRMSE and Pearson correlation values to determine whether the SAW-FMG model's performance differed at 10°/s versus 20°/s. The resulting analysis showed no difference in the main effect for speed for both NRMSE (Φ=0.162, π=0.702) and Pearson correlation (Φ=3.855, π=0.097). These findings suggest that the model's accuracy and linear fit remain consistent across the tested angular velocity range. The lack of significant differences implies that separate calibrations or model adjustments are not necessary for these two speeds. However, certain individuals did exhibit minor differences in consistency between 10°/s and 20°/s. Future investigations may include higher angular velocities or alternative muscle groups to evaluate whether the SAW-FMG approach maintains its robustness under broader dynamic conditions.

Compared with existing research that utilizes various sensor modalities and modeling methods, the approach presented in this study achieves error levels consistent with prior work under isometric conditions but exhibits higher deviation in isokinetic tasks. For instance, Zhou et al. combined EMG with A-mode ultrasound and an SVM regression model to estimate elbow torque at 60°/s under isokinetic conditions, achieving 9.6% NRMSE with the combined dataset, outperforming ultrasound-only (11.70%) and EMG-only (13.64%) approaches. (Y. Zhou, J. Liu, J. Zeng, K. Li, and H. Liu, “Bio-signal based elbow angle and torque simultaneous prediction during isokinetic contraction,” Science China Technological Sciences, vol. 62, no. 1, p. 21-30, December 2018). Alvarez et al. used soft strain sensors to estimate knee torque during 30°/s isokinetic knee extension, reporting an NRMSE of 15% for peak torque comparison with an isokinetic dynamometer.

Jin et al., using a wearable A-mode ultrasound sensor and a quadratic fit, compared their estimates to an isokinetic dynamometer and achieved less than 7.6% NRMSE for both elbow and knee torque estimation during isokinetic contractions at 60°/s, 90°/s, and 120°/s. By contrast, in the current study's isokinetic trials, the group model showed NRMSE values noticeably higher than those reported in the aforementioned literature, especially at lower angular velocities. Such discrepancies may be attributable to differences in sensor modalities, placement, modeling approaches, and specific experimental protocols, particularly under isokinetic conditions. Collectively, these works underscore that, while the present method's performance aligns with established ranges in isometric tasks, further refinements are required to match the accuracy of prior isokinetic studies. In particular, improvements may include adopting more advanced estimation models (e.g., machine learning), expanding the subject pool to enhance model generalizability, and optimizing the structural design and placement of the device.

These findings indicate that, while the SAW-FMG system 100 and the resulting model can still track torque at both speeds, errors increase compared to isometric settings. The results confirm that a single-sensor SAW-FMG approach remains feasible for elbow flexion tasks at slower velocities, though fidelity decreases as motion speed intensifies. The observed errors of about 24-25% at these speeds exceed the 7-15% reported in some previous studies, likely due to the more complex sensor arrays (e.g., multiple strain gauges or force-sensing resistors, EMG) and advanced algorithms (e.g., machine learning) those studies utilized. By contrast, the present method uses a single SAW-FMG sensor 205 and a relatively simple polynomial model, applying an isometric calibration to an isokinetic task without explicitly accounting for the force-velocity relationship established in earlier research. Prior work has shown that concentric muscle force capacity decreases as velocity increases, so the same muscle activation generates different torque outputs at 10°/s versus 20°/s. Additionally, as speeds increase, participants may experience a learning curve when exercising on the machine, potentially introducing greater variability in the measured torque. Integrating this relationship into future models may help reduce RMSE. Moreover, different joints can yield varying error magnitudes owing to distinct biomechanical and anatomical factors, which helps explain variability among studies.

The SAW-FMG sensor system 100 of the present disclosure is noninvasive, straightforward to mount, and may be integrated into wearable systems more easily than ultrasound or electromyography (EMG). The SAW-FMG sensor system 100 focuses on actively contracting muscles (predominantly the biceps brachii during supinated elbow flexion) but may not account for relaxed-arm motion or contributions from other elbow flexors like the brachialis or brachioradialis and antagonists such as the triceps brachii. Additionally, while the elbow is generally described as a hinge joint, it actually involves a more complex articulation of the humerus, ulna, and radius.

The torque estimation model of the present disclosure used two inputs, the SAW signal and joint angle, such that an additional sensor such as a goniometer or an IMU may be used to capture the angle. In embodiments, the SAW-FMG device system 100 may integrate angle sensing into a single device. In embodiments, normalized torque values, calculated relative to an individual's maximum voluntary contraction, may be utilized, which is advantageous for monitoring fatigue and progressive muscle changes (e.g., in multiple sclerosis or Parkinson's disease). However, for applications demanding precise torque values, absolute torque estimation may be a key such that the device may include additional sensors or improved calibration methods to accurately capture the joint angle and muscle force relationship in real-time.

In embodiments, One limitation of the monolithic 128° YX-cut lithium niobate (LiNbO₃) substrate 204 used in the SAW-FMG sensor 100 is mounted on a rigid copper bar 207, which may be uncomfortable for extended use. In embodiments, other materials may be used. In embodiments, it has an inherently high temperature coefficient of delay (TCD), which may also cause discomfort. In embodiments, the monolithic 128° YX-LN may be replaced with lithium niobate on insulator (LNOI) substrates and other mounting materials or options may be implemented. In embodiments, by combining a thin 128° YX-LN layer with underlying SiO2 and Si layers and better flexible mounting structures, the device may be more comfortable and the TCD is significantly reduced.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A force myography system comprising:
a surface acoustic wave sensor,
an SMA connector operable to provide an output of the surface acoustic wave sensor; and
an adjustable band configured for removable attachment to a user, wherein the acoustic wave sensor is mounted on the adjustable band such that movement of a muscle of a user is indicated by the output of the surface acoustic wave sensor.

2. The force myography system of claim 1, wherein the surface acoustic wave sensor comprises;
a substrate including a first interdigital transducer and a second interdigital transducer; and
a first open reflector and a second open reflector mounted on the substrate,
wherein the SMA connector is operably connected to one of the first interdigital transducer and a second interdigital transducer.

3. The force myography system of claim 2, wherein the substrate comprises a 500 μm thick, 128° YX-cut LiNbO₃ wafer.

4. The force myography system of claim 2, wherein the first interdigital transducer and second interdigital transducer comprise chromium and gold.

5. The force myography system of claim 2, further comprising a conducting bar, wherein the substrate is mounted on the conducting bar.

6. The force myography system of claim 5, wherein the conducting bar comprises a 0.5 mm copper bar.

7. The force myography system of claim 2, further comprising a terminal pad electrically connected to the first interdigital transducer.

8. The force myography system of claim 2, wherein the first interdigital transducer, second interdigital transducer, first open reflector and second open reflector are positioned in the substrate such that the first interdigital transducer uses an inverse piezoelectric output to convert electric signals into surface acoustical waves on the substrate which propagate along the substrate until they are reflected by the second interdigital transducer, the first open reflector and the second open back to the first interdigital transducer, wherein strain applied to the substrate by the muscle of the user delays receipt of the reflected waves at the first interdigital transducer corresponding to the torque applied by the muscle.

9. The force myography system of claim 1, wherein the output of the surface acoustic wave sensor indicates a delay caused by the strain introduced by movement of the muscle.

10. The force myography system of claim 1, wherein the output of the surface acoustic wave sensor is processed using a 2D polynomial model to provide an estimate of torque provided by the muscle.

11. The force myograph system of claim 1, further comprising a controller operable to receive the output of the surface acoustic wave sensor and provide an indication of torque provided by the muscle.

12. The force myography system of claim 11, further comprising a wireless transmitter connected to the SMA connector and operable to transmit the output of the surface acoustic wave sensor wirelessly to the controller.

13. The force myography system of claim 11, wherein the controller is a processor provided in a mobile electronic device.

14. The force myography system of claim 11, wherein the controller is a computer system connected to a communications network.

15. The force myography system of claim 11, wherein the controller implements a 2D polynomial model to provide an estimate of torque provided by the muscle.

16. The force myography system of claim 11, further comprising memory operably connected to the controller and configured to store the output of the surface acoustic wave sensor.

17. The force myography system of claim 16, wherein the memory further comprises controller executable code that when executed by the controller implements a 2D polynomial model to provide the indication of torque provided by the model based on at least the output of the surface acoustic wave sensor.

18. The force myography system of claim 17, wherein the indication of torque and the output of the surface acoustic wave sensor are stored in the memory.

19. The force myography system of claim 17, wherein the controller is operably connected to a display and displays the estimate of torque and the output of the surface acoustic wave sensor.
